(19) United States
(12) Patent Application Publication
(10) Pub. No.: US 2005/0032208 A1
(43) Pub. Date: Feb. 10, 2005

(54) Materials and methods to produce stem cells

(76) Inventors: Steve Kah Weng Oh, Singapore (SG); Andre Boon Hwa Choo, Singapore (SG)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/869,393

(22) Filed: Jun. 16, 2004

Related U.S. Application Data
(60) Provisional application No. 60/479,423, filed on Jun. 18, 2003.
(60) Provisional application No. 60/502,744, filed on Sep. 12, 2003.

(51) Int. Cl. C12Q 1/68; C12N 5/08; C12N 15/85

(52) U.S. Cl. 435/366; 435/455

(57) ABSTRACT

A method of generating stem cells, such as human and mouse embryonic stem cells, from a stem cell culture comprising the step of perfusing the stem cell culture by flowing culture media through the stem cell culture and apparatus for generating stem cells are disclosed. Also disclosed are methods and apparatus for generating stem cells, such as human and mouse embryonic cells, by culturing the cells on a hydrophilic plastics membrane. Further disclosed is a supplemented conditioned medium for obtaining improved yields of stem cells, such as human and mouse embryonic stem cells.

### CROSS REFERENCE TO RELATED APPLICATIONS

This application claims priority to U.S. Provisional Application 60/479,423 and 60/502,744 filed Jun. 18, 2003 and Sep. 12, 2003 respectively. Each of the above-identified applications is incorporated herein by reference.

### FIELD OF THE INVENTION

The present invention concerns materials and methods relating to stem cell culture. Particularly, but not exclusively it relates to a method of generating stem cells from a stem cell culture by perfusing the stem cell culture with a flow of culture media, to apparatus for generating stem cells and to uses of the generated stem cells.

### BACKGROUND TO THE INVENTION

Embryonic stem (ES) cells are derived from the inner cell mass of the blastocyst. ES cells have the potential for regeneration of many tissue types for use in cell transplantation and drug discovery. Research on ES cells has been extensive in the past few decades. However, the issue of large-scale production of ES cells remains technically challenging.

In vitro, cells are usually grown in culture media in a batch mode and thus are exposed to non-physiological conditions. Existing techniques for the production of ES cells are centred on static culture of ES cells wherein the ES cells are dish-cultured in monolayers and maintained in contact with a surrounding culture medium, the waste media being replaced periodically. One characteristic shortcoming of such a regimen is the lack of homeostasis due to temporary gradients of metabolic waste products, growth factors and nutrients in the surrounding partially consumed media.

Previous perfusion studies in mammalian systems such as Chinese hamster ovary (CHO) cells¹, chondrocytes and hepatocytes⁸ have been made. However, these currently require complex cell immobilization techniques such as the use of scaffolds⁸. Continuous perfusion systems developed for haematopoietic stem cells (HSC) are described in U.S. Pat. No. 5,605,822 and U.S. Pat. No. 5,763,266.

U.S. Pat. No. 5,320,963 describes a bioreactor for perfusion culture of suspension cells. U.S. Pat. No. 5,605,822 describes a bioreactor system, employing stromal cells to provide growth factors, for growth of HSC cells in culture by perfusion. U.S. Pat. No. 5,646,043 describes growth of HSC cells by continuous and periodic perfusion including media compositions for growth of HSC cells. U.S. Pat. No. 5,155,035 describes a bioreactor for suspension culture of cells by fluid media rotation. In all of the above systems there are limitations in scaling up the bioreactor volume due to the complex design and control requirements of the bioreactors.

WO 03/004626 describes a process for controlling aggregation of spheroid forming cells, e.g. ES cells, by matrix encapsulation enabling embryoid body generation in a well-mixed system.

WO 01/51616, WO 03/020920, U.S. Pat. No. 5,728,581 and U.S. Pat. No. 6,642,048 describe systems for culturing human pluripotent stem cells, methods for expanding stem cells and media that support the growth of primate pluripotent stem cells.

Thus far, methods or systems developed for large scale culture of anchorage dependent stem cells such as the ES cell have not been developed.

Stem cells have potential as important therapeutic tools¹¹. However, to obtain sufficient stem cells to produce viable therapeutic medicaments, the technical difficulties involved in successful large scale culture of stem cells must be overcome.

### SUMMARY OF THE INVENTION

The present inventor has convincingly shown that perfusion of stem cells dramatically improves cell proliferation compared to cells cultured in static conditions. The perfused stem cells remain largely undifferentiated and are capable of forming a variety of tissue types.

The inventor has provided a bioreactor and methods that can at least double the concentration of ES cells in a novel but simple culture system which can be operated in a continuous or periodic/intermittent manner of feeding. This system is also easily scaleable from a small area of a Petri dish to a large tray such as a Nunc plate. As such the inventor has also provided for volumetric scale up of stem cell culture which avoids the complex requirements and technical difficulties of the prior art.

The ability to stably increase stem cell proliferation in cultures provides a great advantage to all forms of medical research and drug discovery which utilise stems cells. Further, the determination that stem cells can advantageously be cultured using a perfusion system allows for the first time the possibility of scaling up the production of stems cells. The issue of large-scale production of stems cells has in the past proved technically challenging.

The inventors have also shown that stem cells can be effectively cultured to obtain high stem cell yields by culturing on suitable membrane surfaces. Up to a 9-fold increase in stem cell yield has been obtained by perfusion culture of stem cells on a hydrophilic and gas permeable membrane and up to a 6-fold increase when cultured on such membranes in static culture. Membranes for culture may be provided in the form of planar membranes or as bags or pouches formed by the membrane material.

Furthermore, the inventors have provided a conditioned medium capable of increasing the yield of cultured stem cells. The conditioned medium is produced by culturing support cells in a culture medium for a time and under conditions such that the number of support cells in the culture has approximately doubled. Dilution of the culture medium with fresh culture media to obtain an approximate support cell density corresponding to that of the initial culture provides a conditioned medium which can be separated from the support cells and used as a culture media to improve the yield of cultured stem cells, including human embryonic stem cells.

Thus, at its most general, in one aspect the present invention provides materials and methods for proliferating stem cells in a non-static culture medium. This type of non-static culture provides enhanced stem cell proliferation above that achieved in static culture and allows for the first time the large-scale production of stem cells. In other aspects the present invention provides materials and methods for improving the yield of stem cell cultures.

Accordingly, in a first aspect of the present invention, there is provided a method of generating stem cells from a stem cell culture comprising the step of perfusing the stem cell culture by flowing culture media through the stem cell culture. The step of perfusion preferably comprises the supply and removal of culture media to and from the stem cells over a predetermined perfusion period. Perfusion resulting in the permeation and/or suffusion of the culture media with the stem cells, wherein the culture media is allowed to spread over and/or around and/or through the stem cell culture. As a result, during perfusion, the cultured stem cells are bathed in a flow of culture media which is being continuously supplied to, and withdrawn from, the cultured stem cells thus providing the stem cells with an environment of constant composition.

The perfused culture media may be continuously supplied from a fresh source of culture media. Alternatively, culture media removed from the cultured stem cells may be circulated one or a plurality of times to re-perfuse the cultured stem cells. Re-circulated culture media may optionally be modified during re-circulation, e.g. by aeration or controlled introduction of additional nutrients.

The stem cell culture is preferably perfused throughout one or more predetermined time periods.

In one preferred arrangement this may comprise continuous, i.e. without ceasing, perfusion for a single time period, which may be prolonged and last preferably for a time period selected from any of 6 hours, 12 hours, 24 hours, 36 hours, 48 hours, 60 hours, 72 hours, 84 hours, 96 hours, 108 hours, 120 hours, 132 hours, 146 hours or a time period selected from within the range 6 to 146 hours.

Alternatively, a plurality of separate perfusion time periods may be preferred, which may be of the same length or be of different duration, each of which may be separately defined. Adjacent perfusion time periods are separated by an interval in which the continuous supply of culture media is discontinued, i.e. the stem cells are being cultured in a non-perfusion culture. Preferable perfusion time periods may last 15 minutes, 30 minutes, 1 hour, 2 hours, 3 hours, 4 hours, 5 hours, 6 hours or a time period selected from within the range 15 minutes to 6 hours. The intervals between adjacent perfusion time periods may be varied or may remain constant. Preferable interval periods between perfusions may last 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours or a time period within the range 1 hour to 24 hours. Accordingly, the stem cell culture may be perfused several times each day, for example twice, three times, four times, five times or six times each day.

The perfusion flow rate, i.e. the rate of flow of culture media through the stem cell culture during perfusion, may be varied according to the growth characteristics of the cell type being cultured so as to prevent cell detachment. Preferably, the perfusion flow rate is in the range 0.05 to 5 ml/min and more preferably one of 0.05 ml/min, 0.1 ml/min, 0.2 ml/min, 0.3 ml/min, 0.4 ml/min, 0.5 ml/min, 11.0 ml/min or 2.0 ml/min.

Preferably, prior to commencing perfusion of the stem cell culture with culture media, the stem cells are initially cultured in non-perfusion, preferably static, culture for a first culture period. The first culture period may last for 1 day, 2 days, 60 hours, 3 days, 84 hours, 4 days or a time period in the range 1 day to 4 days.

Preferably, following perfusion of the stem cell culture, generated stem cells are collected. Collection may take place at a predetermined time, preferably during any of day 4, 5, 6, 7, 8, 9, 10, 11 or 12 from seeding of the stem cell culture.

Preferably, the stem cells may be perfused for one or a plurality of first perfusion periods followed by one or a plurality of second perfusion periods wherein the first and second time periods are of different lengths. Thus, in one preferred embodiment, stem cells are cultured in non-perfusion culture for an initial first culture period, preferably for two or three days, and are subsequently perfused with culture media according to the present invention every ten hours during day 3 to day 5 and then every four hours from day 6 to day 8. Perfusion during the first perfusion period occurs at a first perfusion flow rate and during the second perfusion period at a second perfusion flow rate. The first and second perfusion flow rates may be the same or different.

In any of the embodiments described the flow of culture media may be pulsed, i.e. an intermittent flow, throughout the duration of the selected perfusion time period.

Preferably, in any of the embodiments described the method further comprises the step of attaching stem cells to a plastic or membrane surface prior to the step of perfusing the stem cell culture. The membrane is preferably gas permeable, crystal-clear and chemically inert. The inventor has found that the perfusion culture combined with growing embryonic stem cells on a plastic surface called petriPERM™ enhances proliferation of ES cells by on average 9-fold more than if they were attached on petri dishes in static culture. These results indicate that a gas permeable, chemically inert cell culture membrane provides the ideal surface on which to grow ES cells in accordance with the present invention. In particular, the inventor has found the petriPERM™, a cell culture dish with a gas permeable base made of bioFOLIE, is an ideal surface for growing ES cells in accordance with the present invention. The petriPERM™ membrane is treated by a process called plasma sputtering to make it hydrophilic. Thus, it is preferable to use a gas-permeable, hydrophilic membrane that the ES cells can grow on.

In certain embodiments the membrane may be provided in the form of a bag or pouch. Stem cells may be attached to one or both of the interior or exterior surfaces of the bag or pouch and perfused with culture media.

Preferably, perfusion culture methods of the present invention generate at least a two-fold increase in cultured stem cells.

The pluripotency of the generated stem cells may be determined by use of suitable assays. Preferably, such assays comprise detecting SSEA-1 antigen, alkaline phosphatase activity assay, detection of Oct-4 gene and/or protein expression and by observing the extent of teratoma formation in SCID mice. Formation of embryoid bodies may also be monitored.

In preferred arrangements of the present invention, stem cells generated and/or cultured comprise:

non-human stem cells e.g. rabbit, guinea pig, rat, mouse or other rodent (including stem cells from any animal in the order Rodentia), cat, dog, pig, sheep, goat, cattle, horse, non-human primate or other non-human vertebrate organism; and/or

non-human mammalian stem cells and/or

non-human embryonic stem cells, preferably non-human mammalian embryonic stem cells, more preferably mouse embryonic stem (mES) cells; and/or

human stem cells, more preferably human embryonic stem cells (hES).

In another preferred arrangement of the present invention stem cells generated and/or cultured comprise:

non-human stem cells e.g. rabbit, guinea pig, rat, mouse or other rodent (including stem cells from any animal in the order Rodentia), cat, dog, pig, sheep, goat, cattle, horse, non-human primate or other non-human vertebrate organism; and/or

non-human mammalian stem cells and/or

non-human embryonic stem cells, preferably non-human mammalian embryonic stem cells, more preferably mouse embryonic stem cells;

and excluding human stem cells and human embryonic stem cells.

In another aspect of the present invention, the use of perfusion culture to generate stem cells from a stem cell culture is provided. Preferably, such use results in high yield of generated stem cells.

In a further aspect of the present invention use of stem cells generated by any of the methods of the present invention in the manufacture of a medicament for use in therapy is provided.

In yet a further aspect of the present invention, a pharmaceutical composition comprising stem cells generated by any of the methods of the present invention, or fragments or products thereof, is provided. The pharmaceutical composition useful in a method of medical treatment. Suitable pharmaceutical compositions may further comprise a pharmaceutically acceptable carrier, adjuvant or diluent.

In another aspect of the present invention, stem cells generated by any of the methods of the present invention may be used in a method of medical treatment, preferably, a method of medical treatment is provided comprising administering to an individual in need of treatment a therapeutically effective amount of said medicament or pharmaceutical composition.

In yet another aspect of the present invention, stem cells generated by any of the methods of the present invention may be used to differentiate into another cell type for use in a method of medical treatment.

According to another aspect of the present invention there is provided a method of generating stem cells from a stem cell culture comprising the step of perfusing the stem cell culture by flowing culture media through the stem cell culture wherein the stem cell culture is perfused with culture media for a plurality of time periods.

According to yet another aspect of the present invention there is provided a method of generating stem cells from a stem cell culture comprising the step of perfusing the stem cell culture by flowing culture media through the stem cell culture wherein the stem cell culture is perfused for one or a plurality of first time periods and for one or a plurality of second time periods, wherein said first and second time periods are of different lengths.

According to yet another aspect of the present invention there is provided a method of generating stem cells from a stem cell culture comprising the steps of:

culturing the stem cells in non-perfusion culture for a first culture period prior to;

perfusing the stem cell culture by flowing culture media through the stem cell culture.

According to yet another aspect of the present invention there is provided a method of generating stem cells from a stem cell culture comprising the step of:

culturing the stem cells in non-perfusion culture for a first culture period prior to;

perfusing the stem cell culture by flowing culture media through the stem cell culture;

wherein the stem cell culture is perfused for one or a plurality of first time periods and for one or a plurality of second time periods, wherein said first and second time periods are of different lengths.

According to yet another aspect of the present invention there is provided a method of generating stem cells from a stem cell culture comprising the step of perfusing the stem cell culture by flowing culture media through the stem cell culture wherein said stem cells are not human stem cells.

According to a further aspect of the present invention apparatus is provided for generating stem cells from a stem cell culture by perfusing the stem cells with culture media, the apparatus comprising:

culture media supply means for supplying culture media to said stem cell culture;

culture media removal means for removing culture media from said stem cell culture,

wherein said supply and removal means are arranged to flow culture media through the stem cell culture.

The apparatus preferably further comprises a membrane configured for attachment of one or more stem cells to be cultured.

Preferably, the supply and removal means are arranged to perfuse the stem cell culture with a continuous supply of culture media.

Preferably, the supply and removal means are synchronously operable pumps, the removal means arranged to operate at a higher frequency than the supply means. The resultant perfusion flow rate is preferably in the range 0.05 to 5 ml/min and is configured, using knowledge of the characteristics of the particular cultured stem cells, to prevent detachment of the generated stem cells.

In a preferred arrangement, the apparatus comprises one or a plurality of culture chambers, each containing a stem cell culture, each chamber perfused with culture media supplied and removed by the respective culture media supply and removal means.

In this specification, by stem cell is meant any cell type that has the ability to divide for indefinite periods (i.e. self-renew) and give rise to specialized cells. An embryonic stem cell comprises a primitive (undifferentiated) cell from the embryo that has the potential to become a wide variety of specialized cell types.

Stem cells cultured in the present invention may be obtained or derived from existing cultures or directly from any adult, embryonic or fetal tissue, including blood, bone marrow, skin, epithelia or umbilical cord (a tissue that is normally discarded).

The inventor has shown for the first time that a perfusion bioreactor is technically able to enhance ES cell self-renewal and is suitable for scale up production of ES cells to obtain high yields.

The inventor has provided a perfusion bioreactor to improve stem cell proliferation. This improved culture system provides the cells with an environment of constant composition.

Assays for markers of pluripotency such as surface staining for SSEA-1 antigen, alkaline phosphatase (ALP) enzyme activity, expression of Oct-4 transcription factor, embryoid body formation, and teratoma development in SCID mice were performed on both the perfusion and control cultures. Karyotyping of chromosomes was also made to determine if these cells were normal. The inventors have shown convincingly that ES cells in the perfusion system of the invention proliferate much better than in static conditions, the cells remaining largely undifferentiated and capable of forming a variety of tissue types. The perfusion system is ideally suited for scaling up the production of stem cells, including ES cells, and is thus particularly advantageous, having important utility in the production of very large numbers of ES cells required for use in therapeutic applications. The perfusion method has utility in scaling up both existing and novel stem cell and ES cell lines.

Using the perfusion method of the present invention, the proliferation of mouse ES cells can be enhanced by about two-fold. The perfusion method involves culturing the cells in a bioreactor whereby the cells are first attached to the surface of the bioreactor and media is subsequently fed intermittently or continuously to the culture dishes, and re-circulated back to the feed vessel. In this way, cells can be fed several times a day, and the exchange of media done in a slow and steady manner.

In particular, perfusion culture has been found to increase the number of non-differentiated cells two-fold by either (a) continuous feeding, or (b) by administering more than one feed per day. The ES cells obtained by this method are of high quality and suitable for use directly in therapeutic applications, in the preparation of medicaments for use in therapy, in diagnostic applications or as research tools.

The perfusion culture method provided by the inventors is advantageous in that it provides for large numbers of stem cells to be produced, from which banks of immuno-compatible cells and tissues may be derived.

In yet a further aspect of the present invention there is provided an apparatus for culturing stem cells comprising:
(a) a housing having a wall defining an internal chamber;
(b) a stem cell culture membrane within said chamber and held in position by;
(c) membrane support means; and further comprising
(d) means to supply culture media to said membrane.

The membrane is preferably gas permeable and has a hydrophilic surface for attachment and culture of stem cells. One preferred membrane type is a petriperm™ or biofolie™ membrane.

In the apparatus, the membrane may be substantially planar and may be arranged to extend across the interior space of the chamber such that it is raised from the base of the chamber permitting gas and/or culture media to contact and flow over both surfaces of the membrane. The membrane may be suspended or stretched across the chamber and is preferably supported by attachment to the walls of the housing or to attachment means incorporated therein. The upper membrane surface is preferably configured for stem cell culture.

In one preferred arrangement of the apparatus, the membrane is gas permeable and comprises a first hydrophilic cell culture surface and a second surface, said apparatus further comprising means to deliver a gas supply to said second surface, wherein said means to supply culture media is configured to supply culture media to said first hydrophilic cell culture surface. In this arrangement the first hydrophilic cell culture surface is an upper surface of the membrane.

The housing may comprise a first engagement portion at a first end of said housing and a second engagement portion at a second end of said housing, said first or second engagement portion configured to engage with the other said portion of another said apparatus. In this way a plurality of such apparatus may be connected, e.g. by stacking, such that a larger bioreactor apparatus is provided having a plurality of modules, each comprising a membrane for culture of stem cells. Large scale production of stem cells is thereby provided for. To further increase stem cell generation, each housing may have more than one cell culture membrane, i.e. at least one membrane per housing is provided.

In a further aspect of the present invention a method of preparing a conditioned medium is provided, said method comprising the steps of:
(a) culturing support cells in a culture media;
(b) removing a quantity of the culture media; and
(c) diluting the removed culture media.

The dilution step (c) may comprise dilution with culture media of the kind used in step (a).

The support cells may be embryonic fibroblasts, e.g. mouse embryonic fibroblasts.

The support cells may be cultured in step (a) for at least 12 hours and up to 48 hours and preferably for between 18-30 hours, more preferably between about 22-26 hours and still more preferably for about 24 hours.

The support cells are preferably mitotically inactivated such that they do not grow, i.e. they are growth arrested. This may be achieved by the further step of treating the support cells with mitomycin or another suitable cellular growth inactivation agent. Such treatment step may be performed before step (a) or may be part of step (a), e.g. by including the growth inactivation agent in the culture media.

The support cells may be cultured at a density of 2-5×10⁵ cells/ml, more preferably 2-4×10⁵ cells/ml and still more preferably about 3.5×10⁵ cells/ml.

The method may further comprise the step of:
(d) culturing stem cells in the conditioned medium.

Step (d) may comprise perfusion culture or static culture and may comprise culture of cells when attached to a membrane.

The invention includes the combination of the aspects and preferred features described except where such a combination is clearly impermissible or expressly avoided.

Aspects and embodiments of the present invention will now be illustrated, by way of example, with reference to the accompanying figures. Further aspects and embodiments will be apparent to those skilled in the art. All documents mentioned in this text are incorporated herein by reference.

### BRIEF DESCRIPTION OF THE FIGURES

FIGS. 1A and B illustrate a schematic arrangement of components of the perfusion bioreactor.

FIG. 2—Comparative morphology of cells in perfusion and static culture at day 4, 6 and 8.

FIG. 3—FACS analysis of cultured cells. Scattered plots show the size and granularity of the cell populations. R1 indicates the percentage of gated cells.

FIG. 4—Graphic illustration of total live cell number and percentage viability profiles for perfusion and static cultures. Bar chart represents cell numbers, line graph represents percentage viability.

FIG. 5—(A) SSEA-1 staining of mouse ES cells; (B) ALP staining of mouse ES cells; (C) Embryoid body formation at days 4, 5 and 6 after seeding of cells taken from perfusion culture; (D) Normal karyotyping of mouse ES cells, 40 chromosomes.

FIG. 6—Flow cytometric histograms of SSEA-1 expression on static and perfusion cultured ES cells at day 0, 4, 6 and 8. M1 indicates the percentage of positively stained cells.

FIG. 7—Bar chart illustrating alkaline phosphatase activity of perfusion and static cultures, error bars indicate the standard deviation.

FIG. 8—Appearance of embryoid bodies in ES cells, (A) at the start of the experiment, (B) in static culture, and (C) perfusion culture. All magnification 4×.

FIG. 9—Flow cytometric histograms of Oct-4 protein expression in static and perfusion cultured ES cells at day 0, 4, 6 and 8. M2 indicates the percentage of positively stained cells.

FIG. 10—Expression of Oct-4 detected by RT-PCR. Actin and Oct-4 bands are observable for both perfusion and static cultured ES cells.

FIG. 11—Comparative analysis of tissues in teratomas formed in SCID mice from ES cells after proliferation in (A) static culture and (B) perfusion culture.

FIG. 12—Plot of cell densities obtained on Day 6 for all 3 sets of experiments comparing the petri dish to pertriperm dish cultures in static and perfusion modes. Petriperm™ cultures were average values of duplicate runs.

FIG. 13—A typical growth performance of mES cells in a petri dish compared to petriperm™ dish cultures in static and perfusion modes. Cells were counted on days 4 and 6 and viability was maintained well above 90% throughout each experiment.

FIG. 14—Cell density obtained for all 5 conditions. The values obtained were averages from duplicate runs.

FIG. 15—Comparison between a picture of a creased area (upper left) and that of a smooth area in the same bag (upper right).

FIG. 16a—Quantitative measurement of Oct-4 and SSEA-1 expression in mES cells at the end of perfusion experiments compared to static cultures on petriperm™ (Day 6). SSEA-4 and SSEA-3 were used as the isotype negative controls for Oct-4 and SSEA-1 measurement respectively.

FIG. 16b—Quantitative measurement of Oct-4 and SSEA-1 expression in mES cells at the end of perfusion experiments compared to static cultures on petribags (Day-6). Again, SSEA-4 and SSEA-3 were used as negative control for Oct-4 and SSEA-1 measurement respectively.

FIG. 17—Pictures of embryoid bodies, taken at 40× magnification, formed from cells harvested from perfusion cultures of mES cells grown on petriperm dishes and bags.

FIG. 18—Pictures of teratoma tissues from the three germ layers. Cells were harvested from the static and perfusion cultures in petriperm™ dishes and injected into SCID mice.

FIG. 19—Chromosome spreads obtained from perfusion culture of mES cells on petriperm™ dish and is typical of all other runs.

FIG. 20a—Novel prototypes of bioreactors for scaling up mES cells, round version with 50 cm² and 100 cm² areas.

FIG. 20b—Novel prototypes of bioreactors for scaling up mES cells, rectangular version with 50 cm² and 100 cm² areas.

FIG. 21—Comparison of perfusion and static cultures. hES cells grown as colonies in organ culture dishes.

FIG. 22—FACS results of Oct4 protein expression of hES cell colonies grown in organ culture dishes.

FIG. 23—Comparison of perfusion and static cultures, hES cells grown as clumps in organ culture dishes.

FIG. 24—FACS results of Oct4 protein expression of hES cell clumps grown in organ culture dishes.

FIG. 25—Comparison of perfusion and static cultures, hES cells grown as clumps in 6 cm dishes.

FIG. 26—FACS results of Oct4 protein expression of hES cell clumps grown in 6 cm plates.

### DETAILED DESCRIPTION OF THE BEST MODE OF THE INVENTION

Specific details of the best mode contemplated by the inventors for carrying out the invention are set forth below, by way of example. It will be apparent to one skilled in the art that the present invention may be practiced without limitation to these specific details.

The following assays were used as indicators of pluripotency of mES cells: SSEA-1 antigen, alkaline phosphatase (ALP), Oct-4 protein and gene expression and ability to form embryoid bodies (EBs). Karyotyping was used to detect any chromosomal abnormalities of cells in the perfusion cultures and teratoma development in SCID mice measured the ability to form different tissues in vivo.

SSEA-1 is a phenotypic marker of undifferentiated ES cells, as cells differentiate they lose SSEA-1 expression^{2, 9}. ALP activity, another indicator of ES pluripotentiality, is known to decrease with ES cell differentiation^{6, 9}. Oct-3/4 transcription factor is a master regulator of pluripotentiality that controls lineage commitment⁴. Oct-4 is present in cultured pluripotent ES cells but is absent from all differentiated somatic cell types in vitro and in vivo. Leukemia inhibitory factor (LIF) was used for all cultures since embryonic stem cells can proliferate indefinitely in an undifferentiated state in the presence of LIF^{3, 5}. Quantitative measurements of ES phenotypic markers, functional assays and transcription factor expression over a range of LIF concentrations demonstrated a superior ability of LIF to maintain ES cell pluripotentiality at higher LIF concentrations (≧500 pM)^{7, 10}. The LIF concentration used in this study was 500 pM.

Materials and Methods

Bioreactor Set-Up

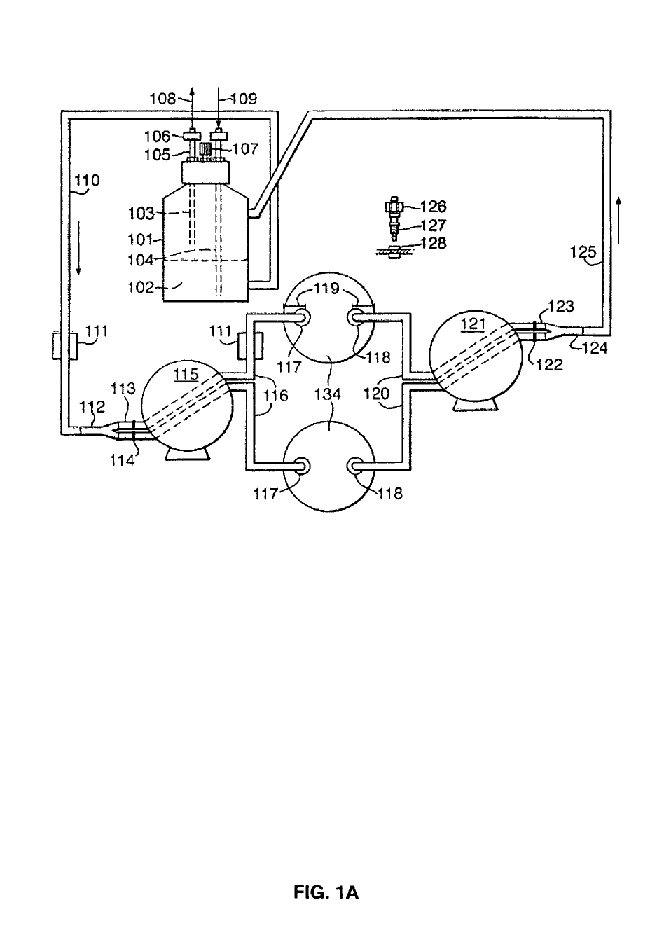

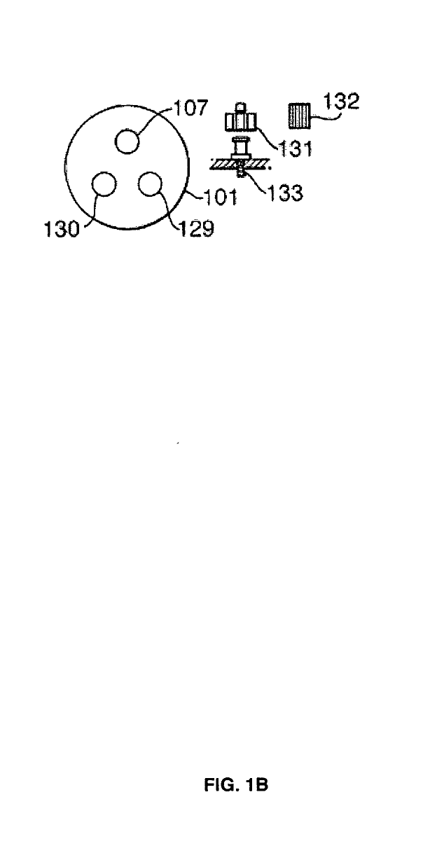

A detailed illustration of the perfusion bioreactor configuration is provided in FIG. 1. Referring to FIG. 1A, the perfusion bioreactor comprises a modified 500 ml Schott Duran bottle 101 having modified ports for air inlet and outlet and containing 100 ml of mES media 102. An air mixture is inlet through a 15 cm long silicon tube 104 (Cole Parmer 06411-63, ID 0.094″, OD 0.156″) extending into the mES culture media 102. Outlet air mixture passes from the container 101 through a 12 cm long silicon tube 103 (Cole Parmer 06411-63, ID 0.094″, OD 0.156″) receiving outlet gas from above the culture media 102. The Schott Duran bottle 101 comprises modified ports for air inlet/outlet wherein a filter 106 (Millex-GP 0.22 μm) is placed in-line with the respective air intake/out-take supply lines and connected to the respective inlet/outlet port by a short length, approx. 1 cm, of tubing 105 (Tygon Laboratory Tubing, Cole Parmer 06409-16). In FIG. 1A, a spare port 107 is unused and is closed off with a screw cap 132 (Qosina P/N 65310).

An air mix is supplied to the inlet port via a supply line 109, the air mix comprising a 10% CO₂/air mix. Outlet air mix is removed via line 108.

Culture media is drawn directly from the base of the Schott Duran bottle 101 via silicon tubing 110 (Cole Parmer 06411-63, ID 0.094″, OD 0.156″). An inlet pump 115 (Masterflex C/L (2 channel) Model 77120-52 (1-6 rpm)) operates to draw the culture media 102 through tubing 110. Inlet pump 115 is connected to the tubing 110 by a Y-joint 112 (Qosina P/N 88404), the dual outlets each connected by 2 cm of silicon tubing (Cole Parmer 06411-63, ID 0.094″, OD 0.156″) 113 to a reducing joint 114 (Cole Parmer 6365-50 1/16×3/32″ reducing joint) connecting to the inlet pump 115.

Inlet pump 115 operates to supply mES culture media 102 to a plurality of culture dishes 134 (Falcon 353002 Tissue Culture Dish, 60×15 mm) via tubing 116 (Masterflex Pharmed Tubing, Cole Parmer 95709-34, ID 1.42 mm) connecting to inlet ports 117.

Perfused culture media is withdrawn from each culture dish 134 at outlet ports 118 via tubing 120 (Masterflex Pharmed Tubing, Cole Parmer 95709-34, ID 1.42 mm) connected to outlet pump 121 (Masterflex C/L (2channel) Model 77120-62 (10-60 rpm)). Removed culture media is then returned via tubing 125 (Cole Parmer 06411-63, ID 0.094″, OD 0.156″) to the Schott Duran bottle 101 for re-circulation. Outlet pump 121 is connected to tubing 125 by reducing joints 122 (Cole Parmer 6365-50 1/16″×3/32″ reducing joint), 1 cm lengths of tubing (Cole Parmer 06411-63, ID 0.094″, OD 0.156″) 123 and a Y-joint (Qosina P/N 88404) 124.

Culture media is passed via the inlet and outlet at each culture dish 134 by respective inlet/outlet ports positioned at the inlet/outlet zone. Each port comprises three components: (i) a head component 126 (Qosina P/N 502201) for connection to the supply/outlet tubing (116, 120), (ii) a media transfer component (Qosina P/N 562321B-1) 127 extending into the culture dish and held in position by (iii) washers 128 either side of the culture dish body.

Referring to FIG. 1B, a plan view of the Schott Duran bottle is shown illustrating the gas inlet and outlet ports (129, 130). A partially exploded view of the port assembly is also illustrated. Each port comprises a media transfer component (Qosina P/N 502201) 133 extending through the bottle inlet/outlet aperture and a connection component 131 for connecting to the tubing 105.

The bioreactor operates inside a 37° C. incubator with a 5% CO₂ supply. Therefore, temperature and pH changes are kept to a minimum. The oxygen level of the culture media was monitored by means of an oxygen and temperature probe inserted into the culture flask via the spare port 107 and connected to a controller. The total volume in the test bioreactor was about 100 ml. Media was circulated to the culture dishes by means of an inlet pump 115, and excess media returned to the culture flask via an outlet pump 121 which operates at a much higher speed than the inlet pump 115. This prevents excess media accumulating in the culture dishes. Two ratchet clamps 111 are available for stopping the media flow through tubings 110 and 116. All cultures were seeded and maintained in the static mode in petri dishes. Two of the cultures were subjected to the perfusion mode of feeding 3 days after inoculation. The controls were left as static cultures and fed once daily. The cultures were maintained for between 4 to 10 days. At days 4, 6, 8 or 10, 1 dish from the controls and 1 dish from the perfusion conditions were sacrificed to determine cell numbers, viability and markers of pluripotency as described below.

Cell Culture

Mouse ES cells were seeded at 6×10⁵ cells per 6 ml volume of petri dish as normal static cultures. CS-1 mES (from Chyuan-Sheng Lin, College of Physicians and Surgeons, Columbia University) were maintained at 37° C. in humidified air with 5% CO₂ in Dulbecco's Modified Eagle's Medium (DMEM) with Glutamax and supplemented with 20% fetal bovine serum (Hyclone), 1% penicillin, streptomycin, L-glutamine and non-essential amino acids (all from GIBCO) and 2-mercaptoethanol. LIF or ESGRO (Chemicon International) was added to complete the culture media immediately prior to use. Culture plates (60 mm tissue culture treated, Falcon) were prepared prior to cell seeding by coating with 0.1% gelatin in phosphate buffered saline (PBS) for 40 minutes. Media was changed daily 3 days after seeding for static cultures, by removing spent media using gentle suction, washing once with PBS and replacing it with fresh media. The feeding of the perfusion cultures was every ten hourly from Day 3 to 5, and every four hourly from Day 6 to 8. The duration of each feeding was 2 hours at a speed of 0.1 ml/min. At different time points, cells from the two different culture modes were trypsinised by incubating with 0.05% trypsin (Invitrogen Life technologies) for 8 mins at 37° C. in humidified air with 5% CO₂. Cell numbers and viability were determined using trypan blue exclusion.

Alkaline Phosphatase (ALP) Assay

ALP activity was determined using a commercially available kit (DG-1245K Sigma). Test cell populations at a concentration of 1×10⁶ cells/10 μl was washed in Hank Buffer supplemented with 2% serum, lysed in 0.1% Triton X for 5 minutes in 96-well NUNC plates before the sample reagent was added. Absorbency readings were taken immediately at 405 nm, 37° C. with a spectrophotometer (Genios, Tecan) over a 5 min period at 1 min intervals. Reaction rate was then calculated using the formula:

$$ALP activity = \frac{ΔA \text{/} min \times Total vol \times 1000 μ mol \text{/} n mol}{18.45 \times Light path ( = 0.193 ) \times No of cells ( ⨯ {10}^{6} )}$$

where ‘ALP activity’ is measured in nMol/10⁶ cells-min.
Flow Cytometry—Cell Surface Analysis

Test cell populations were washed with 1% BSA/PBS (Bovine serum albumin, Sigma) and incubated with a 1:10 dilution of monoclonal anti-SSEA-1 (Developmental Studies Hybridoma Bank) for 30 mins at 25° C. Cells to be analysed for SSEA-1 expression were then washed with 1% BSA/PBS, and incubated with a 1:500 dilution of goat a-mouse antibody conjugated with fluorescein isothiocyanate FITC (DAKO) for 30 mins at 25° C. The cells were then resuspended in 1% BSA/PBS for analysis on a FACScan (Becton Dickinson FACS Calibur). Positive staining was defined as the emission of a level of fluorescence that exceeded levels obtained by >97% of the cells from the same starting population when these were stained with a matched fluorochrome-labeled irrelevant isotype control antibody.

Karyotyping

Test cell populations were reseeded in 60 mm culture plates for 2-3 days before the cells were captured in metaphase by incubating with 0.02 μg/ml colcemid for 1 hour. Cells were then trypsinised and resuspended in 0.076M hypotonic potassium chloride solution for 10 minutes at 25° C. Fixing was done using a fixative containing methanol and acetic acid in the ratio of 3:1. Cells were dropped from a height of about 15-20 cm onto glass slides to cause bursting. Prepared slides were stained using 3%(v/v) Giemsa stain for 15 minutes at 25° C., and viewed under a light microscope.

Embryoid Bodies Formation

Cells to be tested for their ability to form embryoid bodies were cultured in ES media supplemented with 1% methylcellulose (EB media). Test cell populations were suspended in 2 ml of EB media in 3.5 cm non-tissue culture petri dishes at 5000 cells/plate. EBs formed were scored at 6 days using a light microscope.

Intracellular Oct-4 Protein Detection

Test cell populations were washed once with 1% BSA/PBS (Bovine serum albumin, Sigma) before fixing and permeabilisation for 15 min each using a commercially available kit (Caltag Fix and Perm kit). Cells to be analysed for Oct-4 protein expression were incubated with 1 μg equivalent amount of α-Oct-4 monoclonal antibody (COMBI) together with the permeabilisation agent. The cells were then washed twice with 1% BSA/PBS, and incubated with a 1:500 dilution of goat α-mouse antibody conjugated with fluorescein isothiocyanate FITC (DAKO) for 15 mins at 25° C. The cells were then resuspended in 1% BSA/PBS for analysis on a FACScan (Becton Dickinson FACS Calibur). Positive staining was defined as the emission of a level of fluorescence that exceeded levels obtained by >97% of the cells from the same starting population when these were stained with a matched fluorochrome-labeled irrelevant isotype control antibody.

RT-PCR

Total RNA was prepared using the commercially available kit (NucleoSpin RNA II, Macherey-Nagel). 1 μg of the total RNA was annealed with 0.5 μg of oligo dT primer (Promega) at 70° C. for 5 min, followed by reverse transcription at 42° C. for 60 min using ImProm-II reverse transcriptase (Promega). PCR was performed using 5 μl of the RT-reaction mixture for the amplification of Oct-4 and Actin (as a control) in a total volume of 25 μl containing 1 μl of Taq polymerase (Promega, 5 units/μl) and 10 μm of primers. Amplifications were performed for 30 cycles (30 sec at 95° C., 1 min at 55° C., 1 min at 72° C.). Primer pairs for Oct-4 sense were 5′-CGTTCTCTTTGGAAAGGTGTTC-3′ and Oct-4 antisense, 5′-ACACTCGGACCACGTCTTTC-3′. Primer pairs for β-actin sense were 5′-CATCGTGGGCCGCTCTAGGCAC-3′ and β-actin antisense, 5′CCGGCCAGCCAAGTCCAGGACGG-3′. All PCR reactions were analysed by electrophoresis on 1% agarose gel.

Teratoma Formation in SCID Mice

Mouse ES cells harvested from perfusion and static cultures were trypsinised and dissociated into single cell suspensions. 4×10⁶ cells in a 100 μl volume were injected intramuscularly into the right thigh muscle of SCID mice (4 weeks, male). Palpable teratomas were observed after 2 weeks and harvested 4 weeks post-injection. Animals were sacrificed by overdose of carbon dioxide inhalation.

The tissues were fixed in Bouin's fixative for at least 24 hrs followed by dehydration in ascending percentages (50% to 100%) of alcohol, 10-60 mins each, depending on size of tissues. The dehydrated tissues were left overnight in Toluene. Prior to microtome sectioning, the dehydrated tissues are subjected to wax infiltration (60° C.) followed by embedding. The blocks were left to set overnight at room temperature. Sectioning of the tissue blocks is performed at 5-7 μm in thickness. The sections were flattened over warm water (42° C.) before transferring to albuminised glass-slides and left to dry out on a slide warmer.

The sections were stained with Haematoxylin and Eosin dye. Prior to staining, the slides were dewaxed progressively in xylene and alcohol and then hydrated in deionized water. For each of the solvents/reagents, the slides were left for approximately 10s with agitation. The sections were stained first in Haematoxylin dye, which has been filtered, for 15 mins. After 2 washes in deionized water, the slides were left in differentiating fluid (70% alcohol, a few drops of HCl) for 30s, then in tap water for 5-15 mins. After brief rinsing in deionized water, the sections were left in 1-3% Eosin for 5-15 mins. Finally, the sections were dehydrated progressively in alcohol and xylene and dried before sealing with Permount for microscopic examination and classification of the tissues.

Materials and Methods for Perfusion Culture of mouse embryonic stem (mES) cells on petriperm™ membrane

Cell Culture

Five additional sets of experiments were carried out. One set was performed with tissue culture grade petri dishes, 2 sets were performed in petriperm™ (Vivascience; www.vivascience.com) dishes and 2 sets in larger scale petriperm™ bags. The first set of experiments was conducted to determine mES cell densities that could be achieved in conventional static petri dish culture. The second set of experiments was performed to determine the cell densities on static petriperm™ dish. The third set of experiment was then carried out to determine cell densities in perfusion culture on petriperm™ dish. Duplicate runs were conducted for all experiments, and each run was performed with 2 dishes so that one dish could be harvested on day 4 and one on day 6.

After completion of experiments on petriperm™ dishes, experiments were conducted at a larger scale in petribags. The fourth set of experiments was to determine mES cell densities in static culture on petriperm™ bags and the fifth set was to determine cell density for perfusion culture of mES cells on petriperm™ bag. For both sets of experiments, harvesting was done only on Day 6. Duplicate runs were also conducted for these experiments.

For all 5 sets of experiments, the cultures were maintained at 37° C. in humidified air with 5% CO₂. The media used was Dulbecco's Modified Eagle's Medium (DMEM) with Glutamax and supplemented with 20% fetal bovine serum (Hyclone), 1% penicillin, streptomycin, L-glutamine and non essential amino acids and 2-mercaptoethanol (all from GIBCO). LIF or ESGRO (Chemicon International) was added at a concentration of 1 μl/ml media to complete media immediately prior to each use. Culture dishes (tissue culture treated petri dishes and petriperm™ dishes) or culture bags (petriperm™ bags) were prepared prior to cell seeding by coating with 0.1% gelatin in phosphate buffered saline (PBS) for 30 mins. Cells were then seeded at 1×10⁵ cells per cm² of the base area. For petriperm dishes, media was fed to each culture at a feed ratio of 3 ml media per 10⁶ inoculated cells whereas for the petribags, the feed ratio was increased to 5 ml media per 10⁶ inoculated cells. For all the 5 experiments, the media was then changed daily 3 days after seeding. Static cultures were given 1 volume change per day by removing spent media using gentle suction and replacing it with fresh media. Perfusion cultures for petriperm™ dishes were given 4 volume changes per day; 2 volume changes within a duration of 30 mins, twice a day at 12 hours interval. Media was recycled back into a feed bottle containing 32 ml of media for the petriperm™ dishes. A pair of peristaltic pumps was used to achieve this automated feeding and withdrawal of media at flow rates of 8 ml/hr. For the petribags, perfusion was carried out with only 1 vol. change twice daily giving a total of 2 vol. changes per day. The pump flow rate was 50 ml/hr delivered over 1 hr and media was recycled back into a feed bottle containing 300 ml of media for the petribag cultures.

At the point of harvesting, cells were trypsinised by incubating with 0.05% trypsin (Invitrogen Life technologies) for 8 mins at 37° C. in humidified air with 5% CO₂. Cell numbers and viability were determined using trypan blue exclusion using a haemocytometer.

Flow Cytometry-Cell Surface Analysis

Harvested cell populations were washed with 1% BSA/PBS (Bovine serum albumin, Sigma) and fixed using the Fixation medium (Caltag Laboratories) for 15 mins. Later, they were incubated with a 1:20 dilution of monoclonal anti Oct-4 (Santa Cruz Biotechnology) and Permeabilization medium (Caltag Laboratories) for another 15 mins. Concurrently, another test cell population were washed with 1% BSA/PBS and incubated with a 1:10 dilution of monoclonal anti-SSEA-1 (Developmental Studies Hybridoma Bank) for 15 mins. Cells to be analysed for SSEA-1 or/and Oct-4 expression were then washed with 1% BSA/PBS and incubated in the dark with a 1:500 dilution of goat α-mouse antibody conjugated with fluorescein isothiocyanate FITC (DAKO) for 30 mins. The cells were then resuspended in 1% BSA/PBS for analysis on a FACScan (Becton Dickinson FACS Calibur). Positive staining for SSEA-1 was defined as a heterogeneous level of fluorescence while that for Oct-4 was defined as an emission of a level of fluorescence at 100 arbitrary units.

Embryoid Bodies Formation

Cells harvested on Day 6 from each culture were broken down into single cells and 6000 of these cells were seeded into petri dish with 1% methylcellulose in media. For this assay, LIF was not supplemented into the media so that mES cells can differentiate into complex structures called embryoid bodies (EBs) that consist of all the three germ layers.

Teratoma Formation in SCID Mice

If the mES cells maintained their pluripotency, they will produce teratomas consisting of the three germ layers after injection into the severe combined immunodeficient (SCID) mice. Cells harvested from the experiments were injected into the rear leg muscle of the SCID mice. Seven to eight weeks after injection, the resulting teratomas were dissected from the mice, stained and then examined histologically for representatives of the three germ layers.

Karyotyping

A chromosome spread was prepared by swelling cells in a hypotonic solution (0.56% w/v potassium chloride) and then fixing the cells with a mixture of methanol and acetic acid (3:1). These cells are then stained with 3%(v/v) solution of Gurr's Giemsa stain (in Gurr's phosphate buffer), spread on slides, air dried, and observed without a coverslip with phase contrast objectives.

Karyotyping was carried out to confirm that cells remained genetically stable after harvesting. Hence, at least ten sets of chromosome spreads were counted before and after each experimental run. The chromosomal count for mouse cells is 40.

Material and Methods For Perfusion Culture Of Human Embryonic Stem Cells Compared To Control Cultures Without Perfusion

hES Culture Media

Human embryonic stem (hES) cell culture media contained 80%(v/v) DMEM (Invitrogen), 20% (v/v) Hyclone defined FBS (Hyclone), 1×L-glutamine, 1× penicillin-streptomycin, 1× nonessential amino acids, 1× insulin-transferrin-seleniurn (Invitrogen) and 1 mM mercaptoethanol (Sigma).

Preparation of 1× Supplemented Conditioned Media

A conditioned media is obtained by culturing feeder/support cells, e.g. mouse embryonic fibroblasts, in culture media followed by filtering, e.g. with a 2.2 μm filter, to remove the cells leaving the conditioned media containing growth factors and compounds produced by the removed cells.

Mouse embryonic fibroblasts (MEF), named S1, were inoculated at a density of 3.5×10⁵ cells/ml into an organ culture dish. These cells were mitotically inactivated with mytomycin and media added to the culture. After 24 hours, the media was changed and then equilibrated for another 24 hours before collecting as 2× conditioned media. 1× supplemented conditioned media is then made by diluting the 2× conditioned media with an equal volume (50%) of hES media.

For perfusion experiments utilizing organ culture dishes, 5 ml of 2× conditioned media was collected from one T25 flask, plated with 1.75×10⁶ S1 MEF cells. The 2× conditioned media was further diluted by 5 ml of fresh hES media to make up 10 ml of 1× supplemented media, sufficient for one day of perfusion.

2×conditioned medium may be prepared by culturing mitotically inactivated mouse embryonic fibroblasts in culture media at a physiologically suitable temperature and for an amount of time sufficient to obtain a 2×conditioned media enriched in growth factors and compounds produced by the mitotically inactivated cells. The temperature and amount of time will depend on the support cell type selected, typical suitable temperatures will be in the range 32-37° C. For mouse embryonic fibroblasts the time and temperature may be 18-36 hours at 37° C.

Perfusion Apparatus

The setup consists of a 100 ml Duran media bottle supplying fresh 1× supplemented conditioned media to the human ES culture dishes via a low speed peristaltic pump (Cole Parmer). The used media from the culture dishes was transferred into the same bottle via another peristaltic pump. The speed for both the inlet and outlet pumps was set at 0.067 ml/min, which allows media exchange of 2 ml in 30 mins. As the first 1 ml was for purging out the media in the tubing, only 1 ml conditioned media was fed to the cells. The human ES cultures were perfused in a partial continuous manner at 12 hour intervals for 30 mins. 10% CO₂ was bubbled into the media bottle to keep media at pH 6.5-7.0. The perfusion apparatus was placed in the 37° C. incubator together with the human ES cultures during the perfusion experiment. Note that the 1× supplemented conditioned media was replenished daily to prevent degradation of growth factors at 37° C. for long duration.

Human ES Colony Culture

Human ES colonies were passaged by cutting a grown human ES colony into 9 pieces using a glass cutter and then seeded as individual pieces onto organ dishes with feeder layer. The glass cutter was prepared by softening the middle portion of the glass capillary over a flame and then pulled horizontally to approximately 0.25 mm in diameter. The glass tip was broken on a slight angle with no jagged edges to create a smooth cutting edge. Usually 5 human ES pieces were seeded onto one organ dish and that is about 0.8×10⁵ cells. The hES pieces are usually allowed to adhere to the dishes for the first two days before perfusion starts on Day 3. The perfused and static cultures were harvested on Day 7 and 10, where cell count and Oct4 expression level were analysed.

Human ES Clumps Culture

The hES clumps culture was passaged using enzymatic method, where the culture was incubated with collagenase for 7 mins at 37° C. before breaking the hES clumps by gentle pipetting. The hES clumps were then transferred onto culture dishes plated with 1.75×10⁵ S1 mitotically inactivated cells/cm². The media in culture dishes have to be equilibrated 24 hours before use.

The hES clumps were usually allowed to adhere to the dishes during the first two days. Perfusion feeding would start on Day 3 and end on Day 7, where the perfused and static duplicate cultures would be harvested and Oct4 flow cytometric analysis and cell count would be done.

Results & Discussion for Perfusion Culture of Mouse Embryonic Stem Cells on Petridish Cultures

Cell Morphology/Size

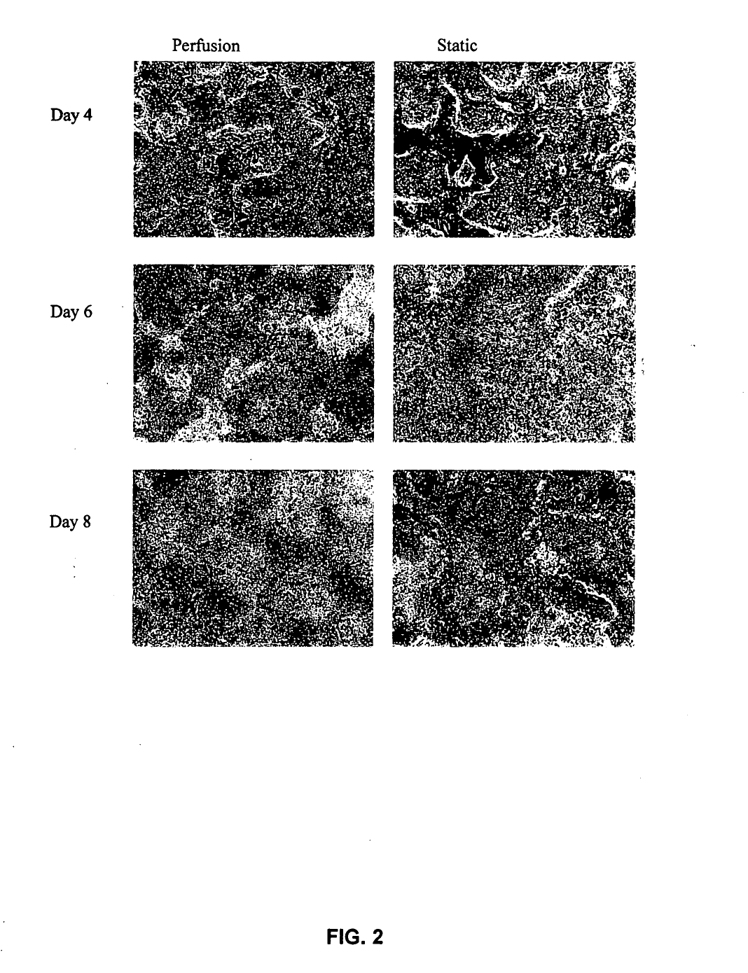

Cells from perfusion cultures appeared more confluent than the static cultures (FIG. 2). From Day 6 onwards, these cells appeared to start stacking on top of one another.

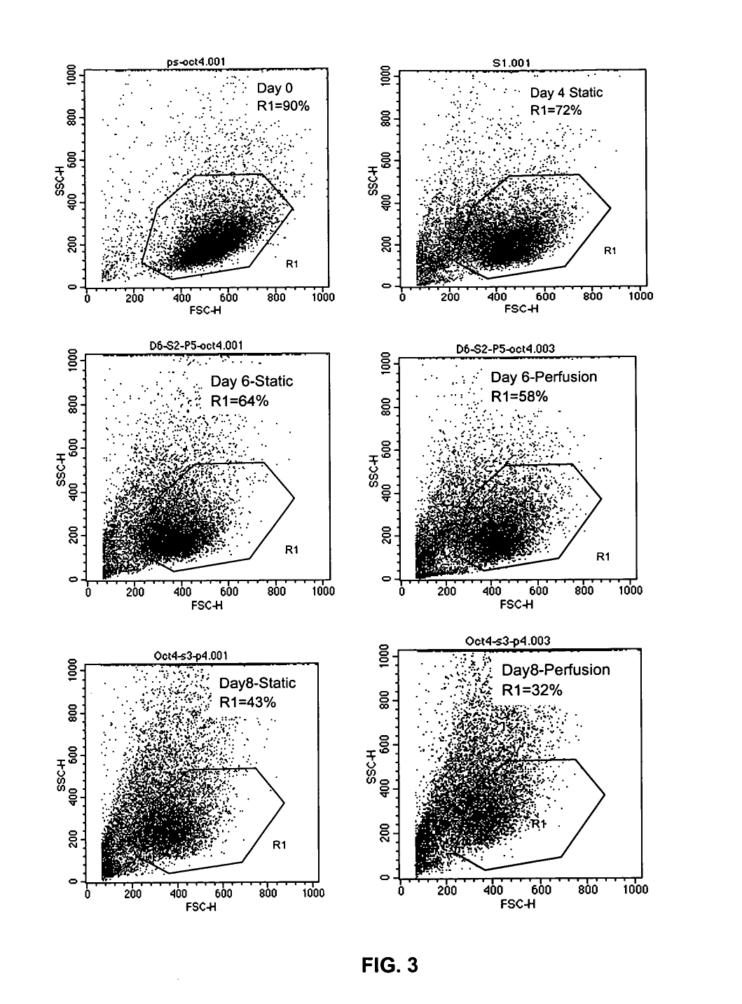

There was a significant shift towards smaller cell sizes, as the cells aggregated, as shown by the dot plots obtained during FACS analysis (FIG. 3). Referring to FIG. 3, the perfusion and static cultures showed similar granularity and cell size. Since the cell size was comparable between the static and perfusion cultures, the mode of perfusion is unlikely to be responsible for this shift.

Cell Numbers/Viability

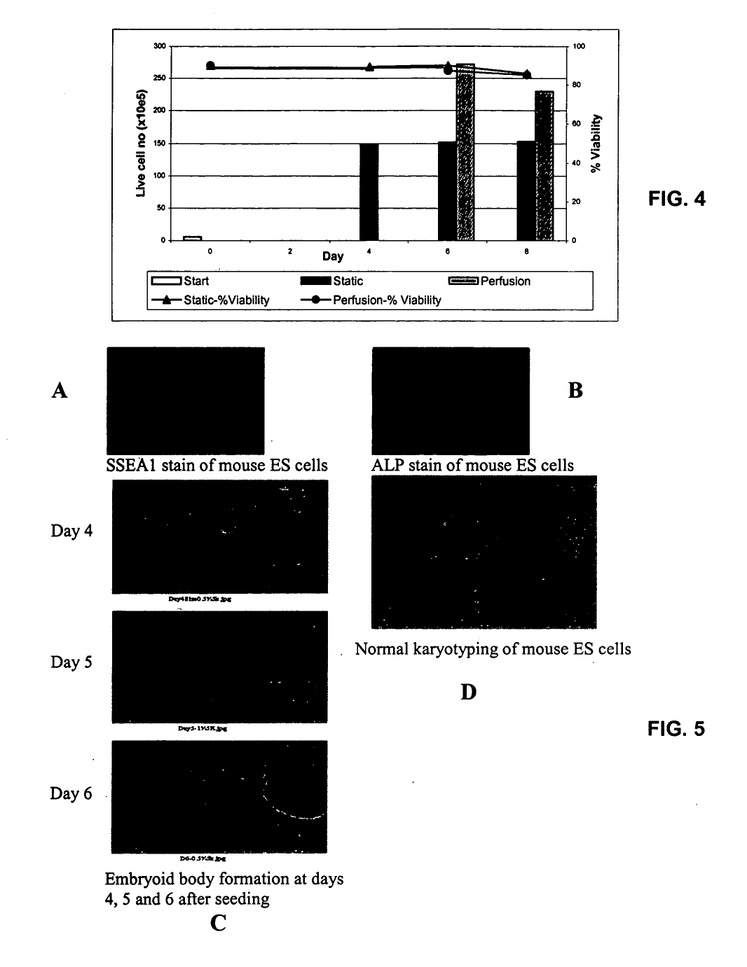

There was little growth in the static cultures after Day 4 (FIG. 4). On Day 6, the perfusion culture cell numbers were 80% more than that of the static culture and even though the live cell number dropped slightly from Day 6 to 8, perfusion cultures retained about 60% more cells than the static ones. Viability remained high for both static and perfusion cultures.

SSEA-1/ALP

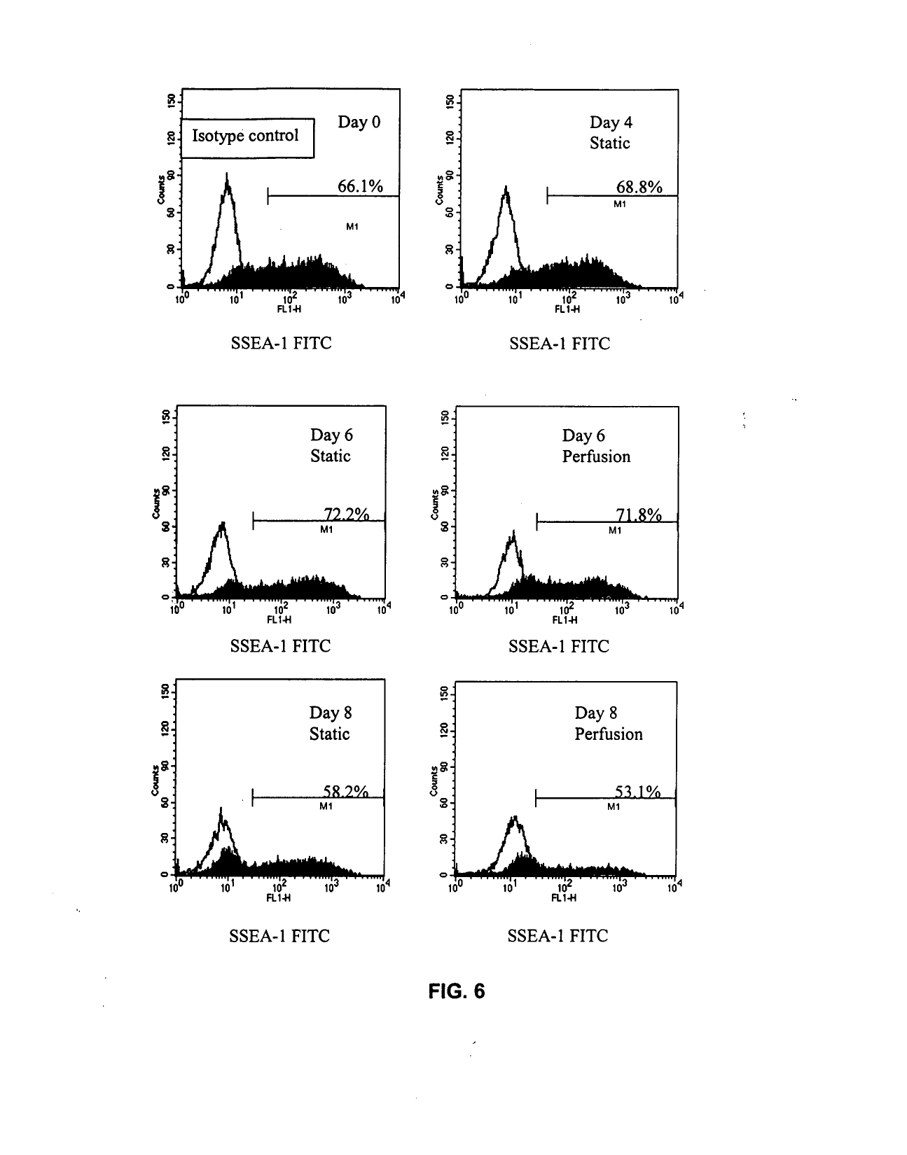

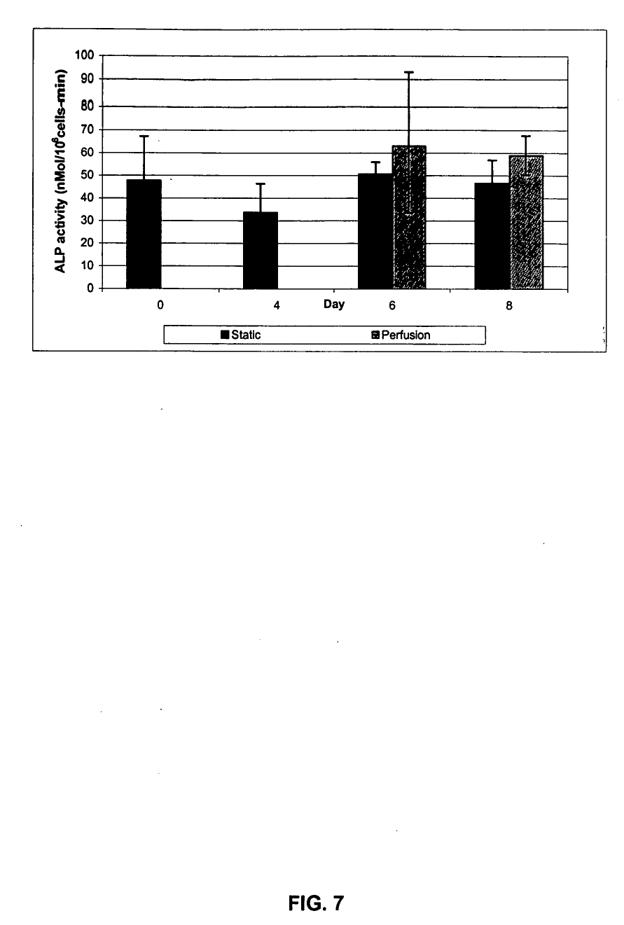

The SSEA-1 results for both static and perfusion remained high until Day 6, but dropped slightly from Day 6 to 8 (FIG. 6). The percentage of cells showing positive SSEA-1 staining was comparable for both static and perfusion cultures at all time points. The alkaline phosphatase activity of all cultures remained approximately the same throughout, taking into consideration the large standard deviation of this assay (FIG. 7).

Karyotyping and Embryoid Bodies

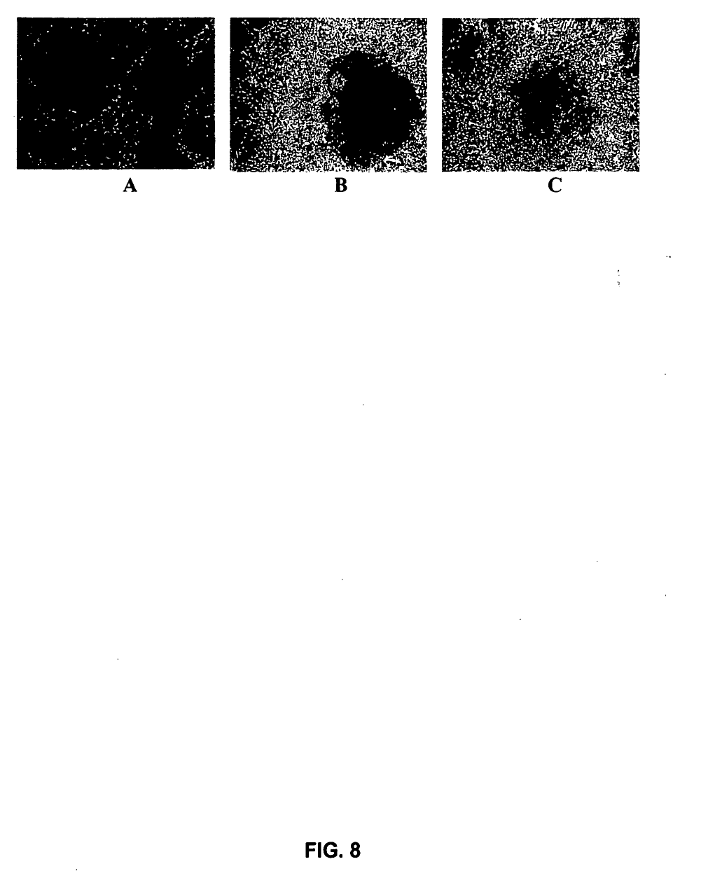

The modal karyotyping count for all the cultures was 40 (Table 1). The range for the counts in perfusion cultures was larger and more unusual counts were encountered. The number of EBs formed from both static and perfusion cultures are shown in FIG. 8. FIG. 8a, b and c show the EBs formed from cells taken from 4, 6 and 8 days of ES perfusion culture. It was observed that the EBs formed from both static and perfusion cultures were smaller on day 8 compared to those at the start. As this assay is purely qualitative, the perfusion cultures demonstrate a similar ability to form EBs as the static cultures.

TABLE 1
Modal count of chromosomes for mouse ES cells
in static and perfusion cultures

| | Start | Static | Perfusion |
|---|---|---|---|
| Mode count of chromosomes | 40 | 40 | 40 |
| Range of counts | 29-41 | 35-42 | 31-78 |
| No. of counts done | 10 | 20 | 20 |

Oct-4 Protein/Oct-4 Gene Expression

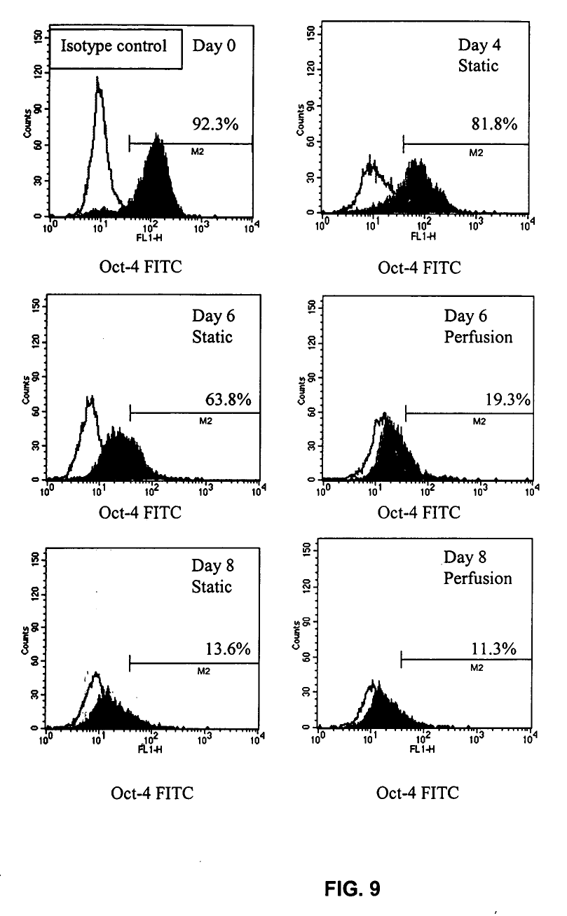

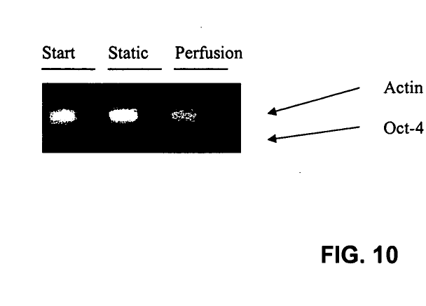

Oct-4 is an excellent indicator of pluripotency. The Oct-4 protein expression levels were high at day 4, but decreased at days 6 and 8 (FIG. 9). Lowest levels of the protein were observed in perfusion cultures and for static cultures on Day 8, thus it may be better to harvest cells at day 6 after the start of the experiment. The RT-PCR results showed Oct-4 bands for both static and perfusion cultures (FIG. 10). Therefore the method of perfusion did not cause the loss of Oct-4 transcription factor.

Teratomas Formation in SCID Mice

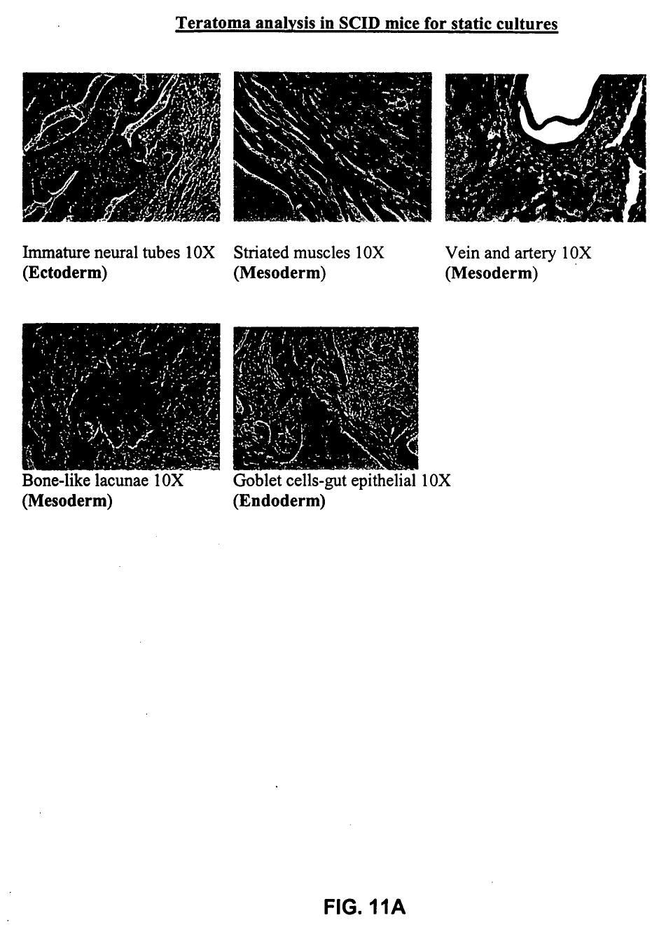

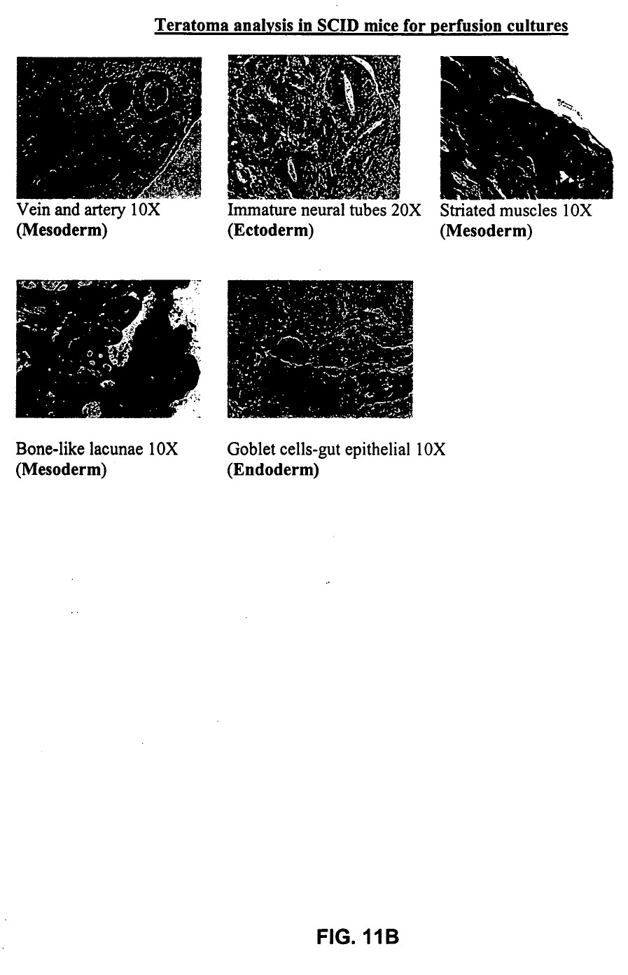

FIG. 11 shows the analysis of a variety of tissues derived from the teratomas formed in SCID mice after 6 weeks representing the ectoderm, mesoderm and endoderm. Examples of an ectodermal tissue are neural tubes, muscles, vein, artery and bone from the mesoderm, whilst epithelial cells come from the endoderm. As can be seen the ES cells produced in static cultures and perfusion cultures are both able to form the diverse tissue types from each layer of cells. Thus the ES cells expanded by perfusion culture are capable of developing to the full range of tissue types.

Summary of separate Perfusion Experiments

As can be seen in Table 2, seven separate independent experiments showed that perfusion culture significantly enhances ES cell growth compared to static culture. Improvement varied from 160% to 340% with an average improvement of 239%.

TABLE 2
Summary of results from mES
perfusion cultures

| | | Maximum | | |
|---|---|---|---|---|
| | | Number of | | Percentage |
| Separate | Conditions of | live cells (×10⁶) | | improvement |
| Expt Runs | culture | Control | Perfusion | (%) |
| 1 | Static, fed once a | 32.7 | 111.2 | 340 |
| | day (day 3-6) | | | |
| | Perfusion, fed 2 x | | | |
| | a day (day 3-6), | | | |
| | fed 4x a day (day | | | |
| | 7-10) | | | |
| 2 | Static, fed once a | 7.2 | 12.6 | 175 |
| | day (day 3-7) | | | |
| | Perfusion, fed 2 x | | | |
| | a day (day 3-7), | | | |
| | fed 4x a day (day | | | |
| | 7-10) | | | |
| 3 | Static, fed once a | 9.4 | 16.4 | 174 |
| | day (day 3-6) | | | |
| | Perfusion, fed 2 x | | | |
| | a day (day 3-6), | | | |
| | fed 4x a day (day | | | |
| | 7-8) | | | |
| 4 | Static, fed once a | 15.3 | 27.2 | 178 |
| | day (day 3-8) | | | |
| | Perfusion, fed 2 x | | | |
| | a day (day 3-5), | | | |
| | fed 4x a day (day | | | |
| | 6-8) | | | |
| 5 | Static, fed once a | 5.6 | 19.2 | 342 |
| | day (day 3-6) | | | |
| | Perfusion, fed 2 x | | | |
| | a day (day 3-8) | | | |
| 6 | Static, fed once a | 7.6 | 18.4 | 242 |
| | day (day 3-8) | | | |
| | Perfusion, fed 2 x | | | |
| | a day (day 3-8) | | | |
| 7 | Static, fed once a | 25.4 | 40.7 | 160 |
| | day (day 3-6) | | | |
| | Perfusion, fed 2 x | | | |
| | a day (day 3-6) | | | |
| Averages | | 14.7 | 35.1 | 239% |

The perfusion reactor performs significantly better than the usual static cultures in terms of cell proliferation. The perfusion cultures also show similar results in important and accepted markers of pluripotency such as SSEA-1, ALP and Oct-4 transcription factor at the protein and mRNA levels. Other key parameters such as cell morphology, viability and chromosomal changes within the cells were found to be similar in the perfusion reactor compared to static cultures. From all these data, the ability of the perfusion reactor to enhance ES cell growth while maintaining their pluripotent capacity is confirmed. Equally important is the fact that such a reactor is technically feasible for scaling up purposes.

The perfusion culture system facilitates the supply of nutrients and oxygen to the cells and the removal of metabolic products. During perfusion, the medium flow rate is important in determining mass transfer rates, and should be set such that it does not cause cell detachment and provides sufficient oxygen supply to the cells. Subjecting cultures to perfusion only on the third day after inoculation and using intermittent feeding patterns instead of the continuous mode eliminated the problem of cell detachment. The medium flow rate of 0.1 ml/min was based on the oxygen uptake rate of the cells. From the above results, perfusion cultures perform similar to the control (static) cultures in terms of markers for pluripotency.

These experiments have shown that mouse embryonic stem cells can be expanded in perfusion cultures. Perfusion cultures over 8 days generated approximately two times more ES cells than those grown on static petri dishes. Biochemical parameters such as SSEA-1 surface antigen, alkaline phosphatase, karyotyping, embryoid body formation were similar to control conditions.

Initial experiments with mouse embryonic stem cells in perfusion cultures over 8 days generated approximately two times more ES cells than those grown on static petri dishes. However, there was a reduced level of Oct-4 expression and cells were smaller at higher cell densities. After changing the perfusion frequency from 4 times to 2 times a day and stopping the experiments on day 6, two times more cells can still be achieved but Oct-4 levels and cell sizes were normal as were the other biochemical parameters, compared to static cultures.

Perfusion culturing of ES cells provides a method for large scale production of ES cells which is suitable for continuous automated operation. Automation of the process 24 hours a day, 7 days a week will facilitate production of large therapeutic quantities of ES cells.

Results and Discussions for Perfusion Culture of mouse embryonic stem (mES) cells on petriperm™ membrane

Cell Growth and Viability

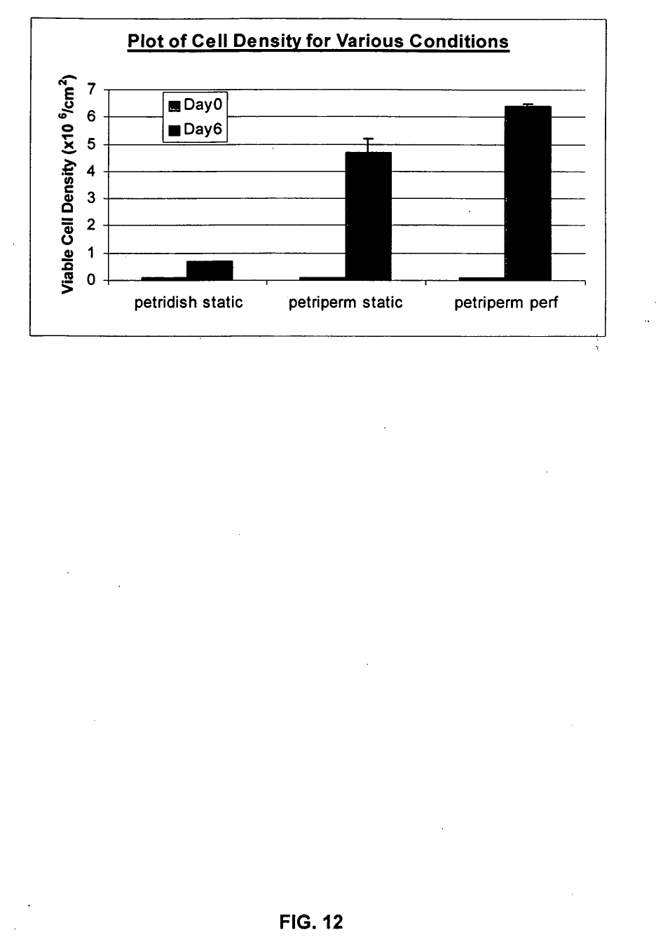

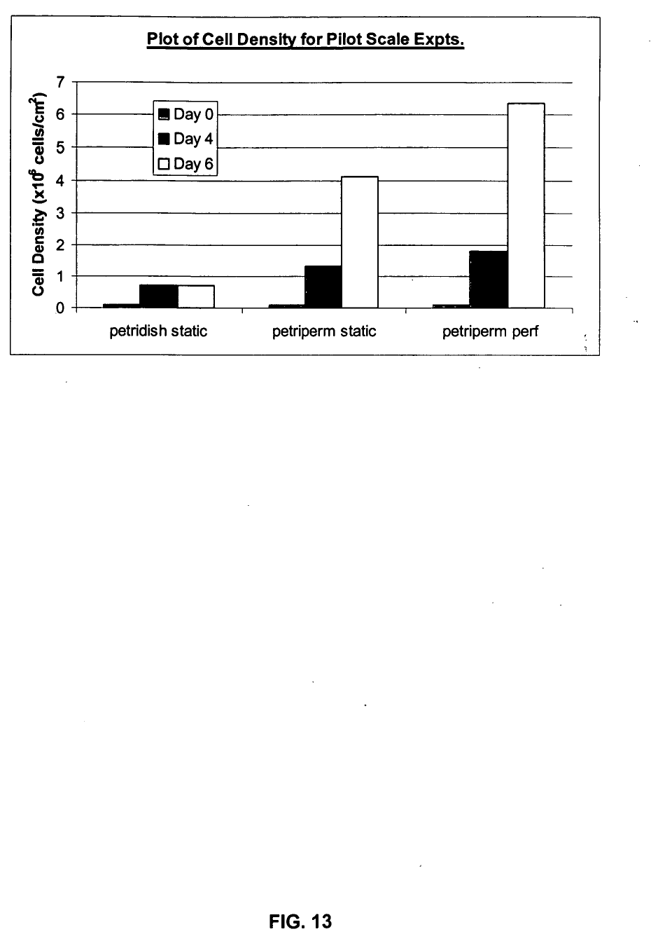

FIG. 12 shows a plot of cell density obtained on day 6 for all 3 sets of experiment on conventional petri dish and petriperm™ dishes. All cultures were seeded with 10⁵ cells/cm². As expected on day 6, static petri dish culture gave the lowest final cell density at 0.7×10⁶ cells/cm², static petriperm™ dishes achieved 4.7×10⁶ cells/cm² and perfusion culture on petriperm™ dishes gave the highest cell density of 6.4×10⁶ cells/cm². Thus petriperm™ dishes enabled a 9-fold increase in cell densities per unit area to be achieved. FIG. 13 shows the typical growth performance of the 3 conditions, it was noted that static petri dish culture could only sustain mES cells growth for up to day 4 while petriperm™ dishes could sustain mES cells growth till the end of the experiments. Viability of the cells for each culture was maintained at above 90% throughout the experiments. The improved cell growth on petriperm™ dishes was attributed to the fact that the petriperm™ membrane used as the base of the dishes is O₂ and CO₂ permeable, thereby allowing optimal mass transfer of these 2 gases. The membrane is also hydrophilic improving cell adhesion.

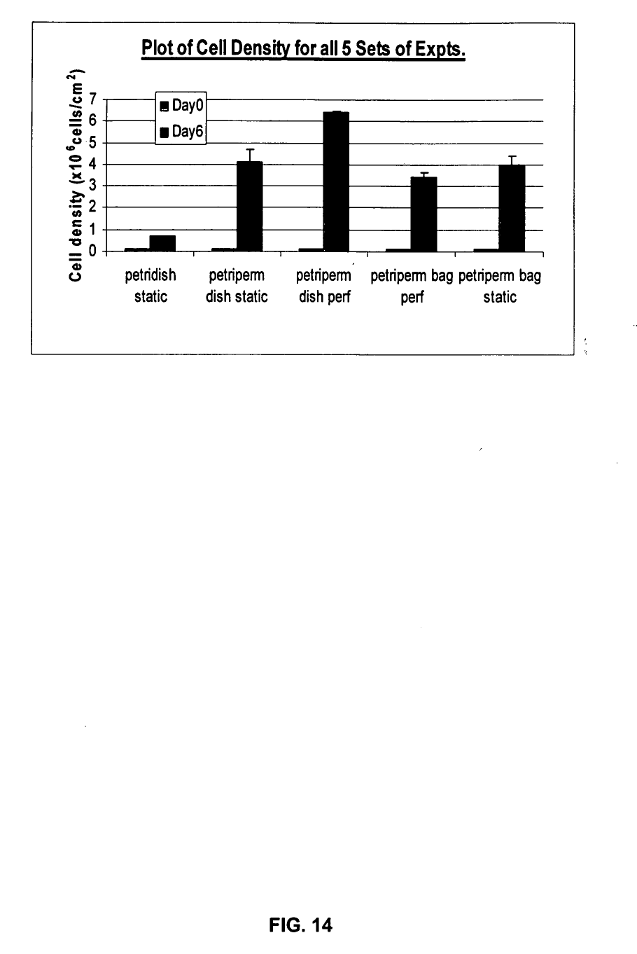

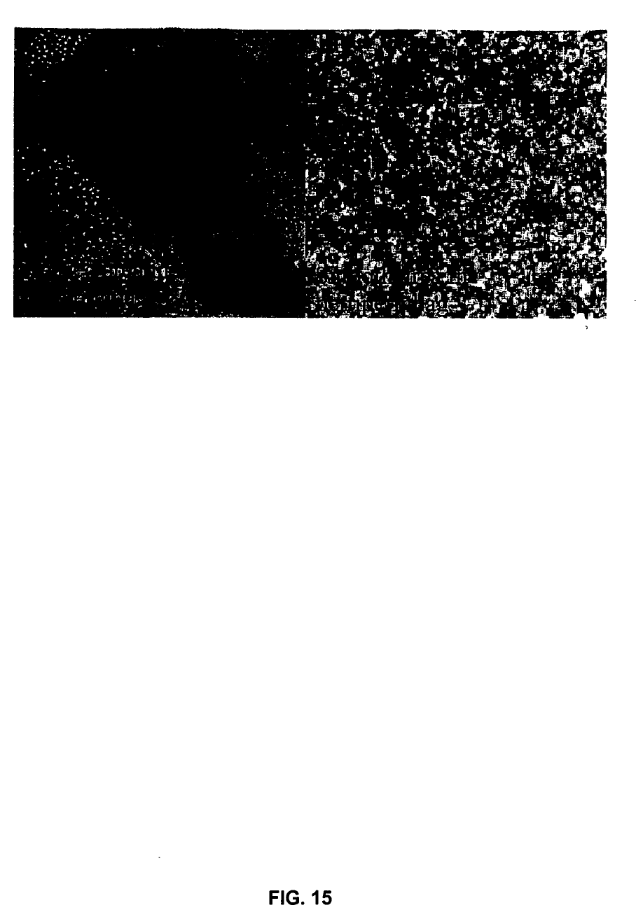

Cell densities obtained for the petribags experiments are shown in FIG. 14. Viability of the cells for each culture was again maintained well above 90% throughout the experiments. The values obtained were an average of duplicate runs and were compared to the previous 3 experiments conducted with petriperm™ dishes. It can be observed that although a more than 9-fold increase in cell density was obtained for the small scale experiments, these results were not directly translated to the scale-up petribags which have an increase surface area of 100 cm², despite increasing the feed ratio and keeping all other conditions constant. Instead, the inventors achieved approximately 5 to 6-fold increases in cell densities (3.3×10⁶ cells/cm² and 4×10⁶ cells/cm²) for both static and perfusion culture on petriperm™ bags compared to 0.7×10⁶ cells/cm² achieved in petri dish cultures. This further indicated that at a feed ratio of 5 ml media/10⁶ inoculated cells, perfusion has no advantage over static culture. From the above observations, it was suspected that nutrients were not the limiting factor, but there might be a change in the intrinsic properties of the petriperm™ membrane during the processing stage to make it into bags. Careful observation showed that the petriperm™ bags were visibly creased after autoclaving, as they were flimsy compared to the petriperm™ dishes. The presence of these creases adversely affected the cell growth and an illustration is included in FIG. 15. The creased areas of the petriperm™ bag were bare while a smooth area in the same petriperm bag was densely packed with cells. This unequal distribution of cells within the bag might have resulted in the lower cell density obtained overall.

Quantitative SSEA-1 and Oct-4 Measurement

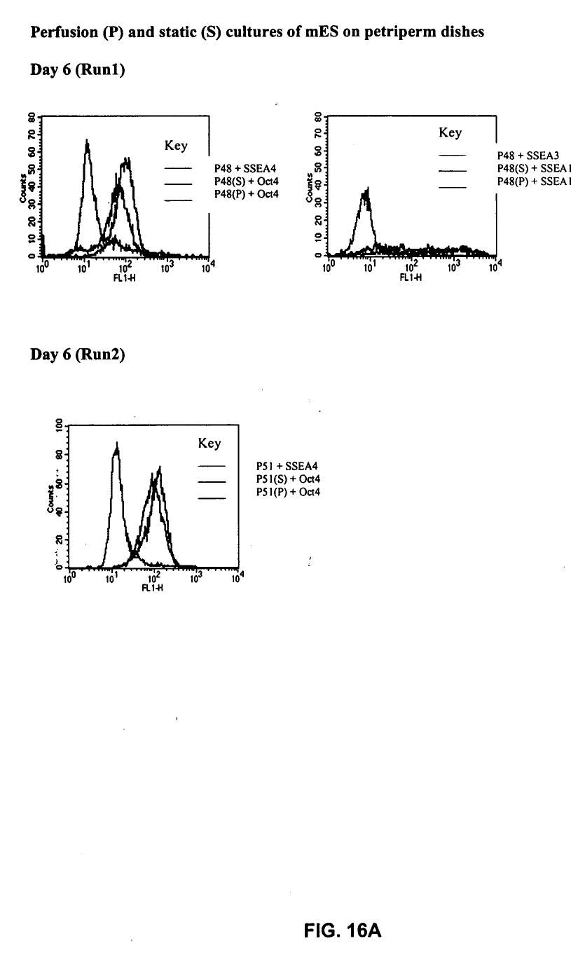

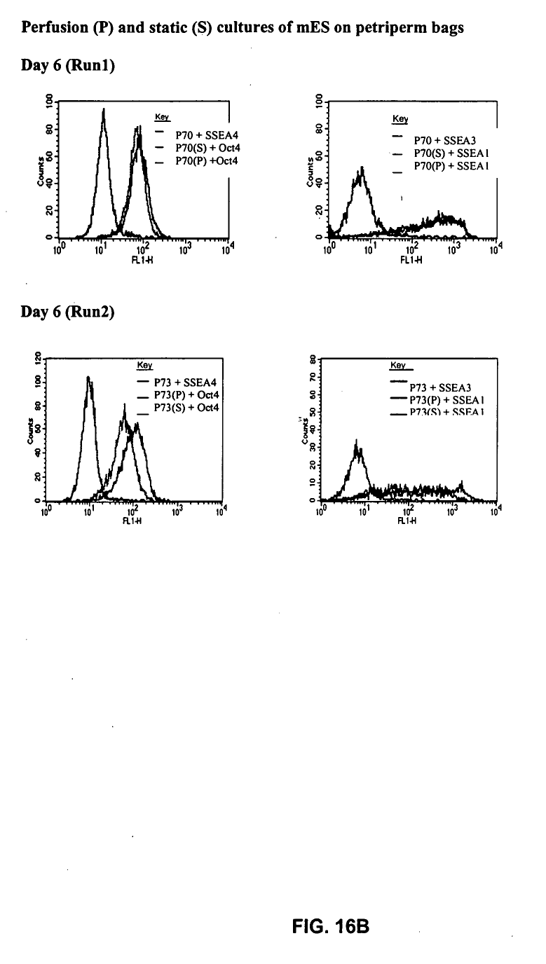

The mES cells were quantitatively stained for SSEA-1 and Oct-4, which are known pluripotent markers of mouse ES cells, on Days 4 and 6 of each experiment and analysed by flow cytometry. As shown in FIG. 16a, fluorescence level of 100 arbitrary units for Oct-4, is strongly indicative of pluripotent ES cells for both experimental run 1 and 2 in the petriperm™ dishes. Oct-4 expression levels are similar for both the static and perfusion cultures. As for SSEA-1 which is an IgM antibody, staining produces a heterogeneous level of fluorescence. It is known that as ES cells differentiate, they lose SSEA-1 expression. Hence, presence of SSEA-1 in all our runs indicates ES cells pluripotency. However, it is known that that SSEA-1 is not as stringent a marker of pluripotency as Oct-4 for ES cells. FIG. 16b also shows that both experiments in petribags produced mES cells which express similar Oct-4 levels in perfusion and static mode. Similarly SSEA-1 expression is maintained in both methods of culture in petribags.

Embryoid Bodies Formation and Teratoma Formation in SCID Mice

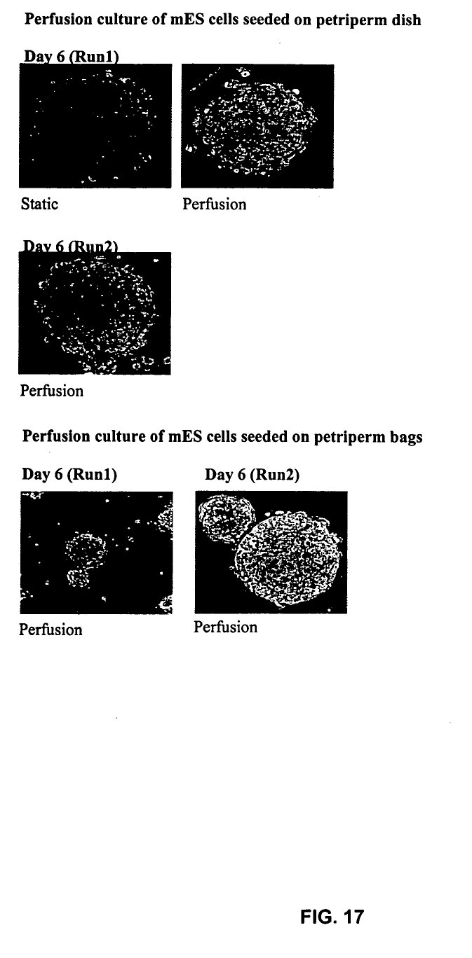

Harvested mES cells from these experiments were seeded into 1% methylcellulose with complete media without LIF and embryoid bodies were identified after 7 days. FIG. 17 shows that embryoid bodies (EBs) can be formed from mES cells harvested from the static and both perfusion cultures taken from petriperm™ dishes upon the removal of LIF. Similarly, mES cells also formed embryoid bodies from petribag cultures. The ability to form EBs is important as this is the intermediate stage towards differentiation into the desired tissue type.

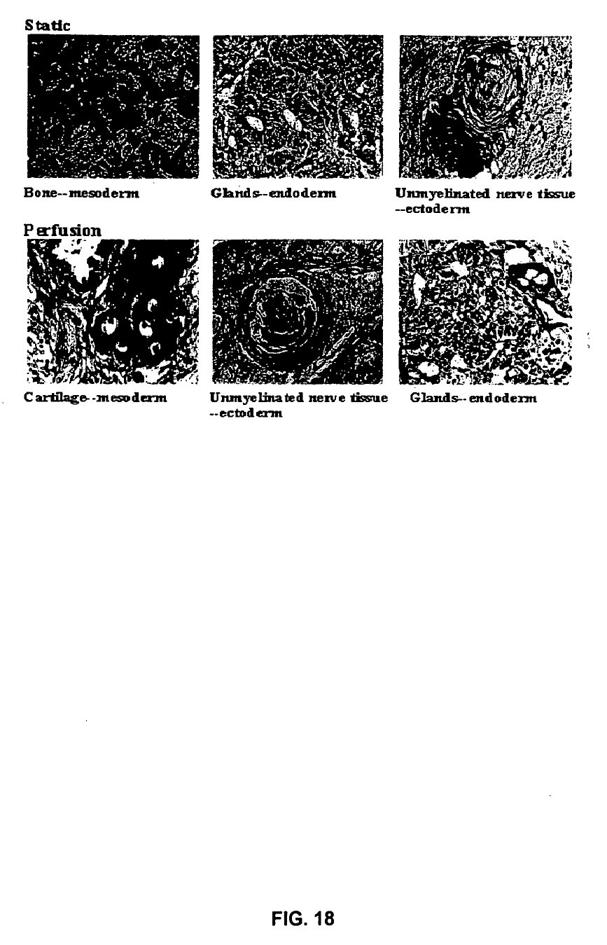

Finally, mES cells taken from petriperm™ static and perfusion cultures were injected into SCID mice to measure the presence of teratomas. Teratomas containing tissues derived from the three germ layers such as bone or cartilage in the mesoderm, glands from the endoderm and nerve tissues from the ectoderm are as shown in FIG. 18,

Thus the results of the EB assays, teratoma formation combined with Oct-4 and SSEA-1 expression provided substantial evidence that our present system did not compromise cell pluripotency while significantly increasing final cell densities by over 9-fold in perfusion cultures on petriperm™ membranes.

Karyotyping

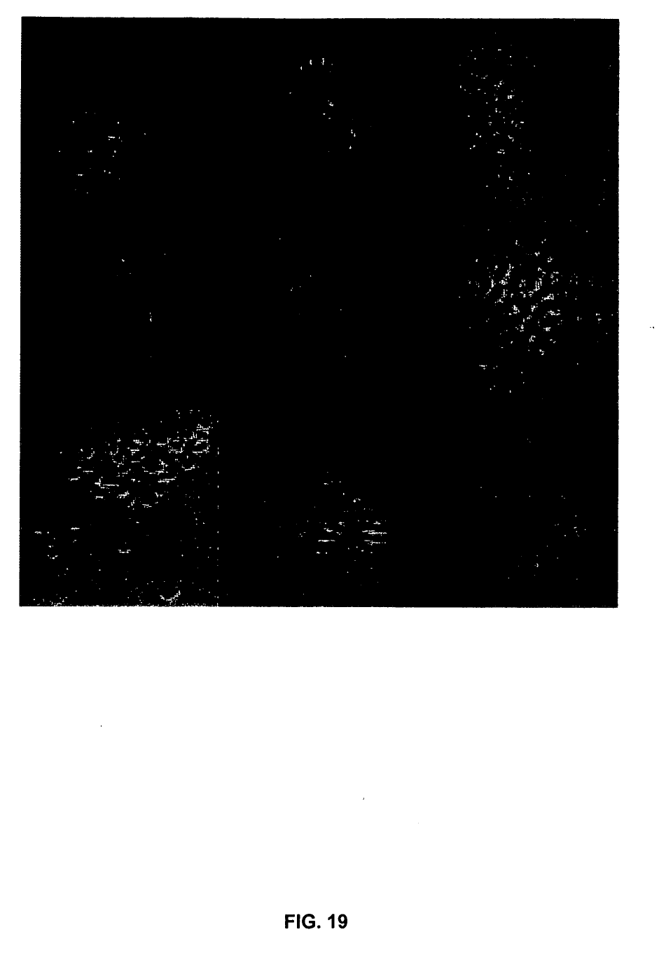

Besides ensuring pluripotency of the cells, karyotyping was carried out before and after each trial run to ensure that cells remain genetically stable. Karyotyping is a valuable research tool used to determine the chromosome complement within cultured cells. It is important to keep in mind that karyotypes can evolve with continued culture. Because of this evolution, it is essential to determine the karyotypic stability of the cultured mES cells. As shown in Table 3, the modal count of the chromosomes for each run was 40, similar to the chromosome count of a typical mouse cell, indicating that the cells harvested remained genetically stable. FIG. 19 shows typical pictures of chromosome spreads that were obtained.

Alternative Prototype Bioreactors for the Scale up of mES Cells

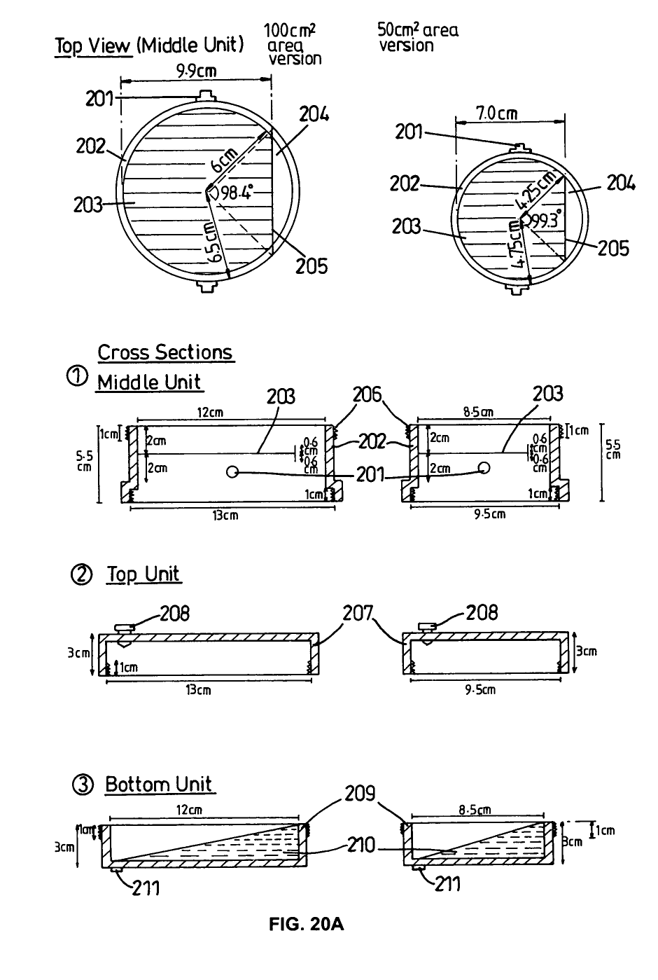

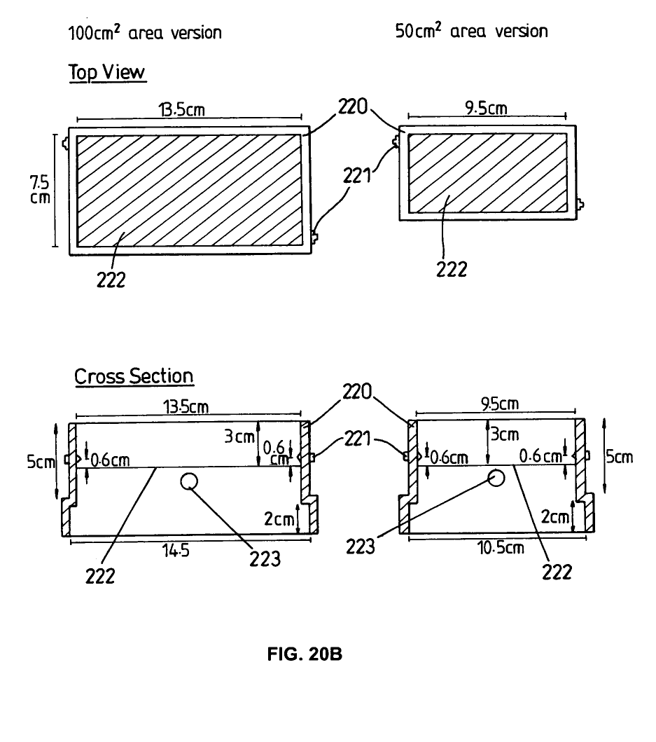

FIGS. 20a and 20b show 2 designs of bioreactors which will allow the use of petriperm™ for the expansion of mES cells. The former is a round version similar to a petri dish format, which can be made with 50 cm² or 100 cm² areas. The latter is a rectangular version also provided with 50 cm² or 100 cm² areas. In both cases, provision is allowed for the stacking of additional layers of these bioreactors to increase the scale and surface area available for cell culture. There are also ports for the passage of air to enable aeration via the permeable petriperm™ membranes at each layer of the bioreactor.

The bioreactor illustrated in FIG. 20a comprises a generally cylindrical structure made up of a top unit, middle unit and bottom unit which may be connected by mating threads formed at corresponding inner and outer circumferential surfaces of the adjacent units. In the embodiment illustrated, each unit has a wall constructed of 0.5 cm thick polystyrene (202, 207, 209).

An inlet (208) is provided in the top unit for the in-flow of culture media.

The middle unit has a generally cylindrical wall formed from 0.5 cm thick polystyrene (202). A petriperm™ membrane (203) is stretched across the central space and is supported at the wall interior such that the membrane is arranged in a substantially horizontal plane. Stem cells may be attached to the membrane and cultured. The membrane is shaped to leave a gap (204) at one side of the chamber, an elongated polystyrene member (205) is situated along the free edge of the membrane to retain culture media on the membrane, thus acting as a weir. Excess culture media is permitted to flow over the weir. Gas inlet and outlet ports (201) are provided on the middle unit below the level of the membrane, which is gas permeable, in order to provide the culture with an appropriate gas supply, e.g. 5% CO₂.

The exterior wall of the upper portion of the middle unit is formed into a male or female thread (206). The wall of the lower portion is formed to be of larger diameter than the upper portion and the interior is formed into the opposing male or female thread relative to the upper portion. A plurality of middle units may thus be secured to each other by engaging the mating threads provided on adjacent upper and lower wall portions. In this way a plurality of middle portions may be stacked to form a single bioreactor, having a single top unit and single bottom unit at opposing ends of the bioreactor. Such an arrangement provides for a plurality of membrane culture surfaces and thus permits scale up of stem cell production.

A polystyrene insert (210) is provided in the bottom unit forming a slope surface directing culture media to an outlet (211).

FIG. 20b illustrates the middle unit of an alternative bioreactor having a box construction, the walls (220) of the bioreactor made of 0.5 cm thick polystyrene and having an inlet and outlet port (221) for inlet and outlet of culture media. The bioreactor walls further provide gas inlet and outlet ports (223) for supply and removal of gas, e.g. 5% CO₂, from the culture.

A petriperm™ membrane (222) is tightly suspended across the interior of the bioreactor above the level of the gas inlet port and below the level of the culture media inlet and outlet ports. The petriperm™ membrane is gas permeable.

The middle unit shown in FIG. 20b permits sliding engagement, e.g. force-fit engagement, of adjacent middle units in order to provide a plurality of stacked middle units in a single bioreactor. The lower wall portion of each middle unit has a larger diameter than the upper wall portion and is arranged to slide over the upper wall portion of an adjacent middle unit or bottom unit.

In operation of the bioreactors illustrated in any of FIGS. 20a and 20b, stem cells are attached to the petriperm™ membrane and the culture media inlet and outlet ports are appropriately connected to culture media supply and removal conduits and, in the case of perfusion culture, to a peristaltic pump. The gas inlet port is connected to a gas supply. Cells may then be cultured for a desired time period and generated stem cells may be collected from the petriperm™ membrane.

TABLE 3
Chromosomal counts of mES in perfusion culture
on petriperm ™ dish and petriperm ™ bag, indicative of
normal karyotypes during culture.

| | Inoculum | Perfusion | Inoculum | |
|---|---|---|---|---|
| | cells at | run 2 on | cells at | Perfusion |
| | the start | petriperm ™ | the start | run 2 on |
| | of | dish | of | petriperm ™ |
| | petriperm ™ | (Cells | petriperm ™ | bag (Cells |
| Conditions | dish | taken from | bag | taken from |
| of culture | experiment | day 6) | experiment | day 6) |
| Count 1 | 40 | 40 | 40 | 40 |
| Count 2 | 37 | 40 | 40 | 43 |
| Count 3 | 40 | 31 | 40 | 40 |
| Count 4 | 40 | 40 | 37 | 40 |
| Count 5 | 40 | 40 | 40 | 40 |
| Count 6 | 40 | 41 | 40 | 40 |
| Count 7 | 40 | 39 | 40 | 43 |
| Count 8 | 38 | 40 | 40 | 40 |
| Count 9 | 40 | 40 | 40 | 40 |
| Count 10 | 36 | 39 | 43 | 40 |
| Modal | 40 | 40 | 40 | 40 |
| count | | | | |

Results And Discussion For Perfusion Culture Of Human Embryonic Stem Cells Compared To Control Cultures Without Perfusion

Perfusion Cultures of hES Cell Colonies on Organ Culture Dish

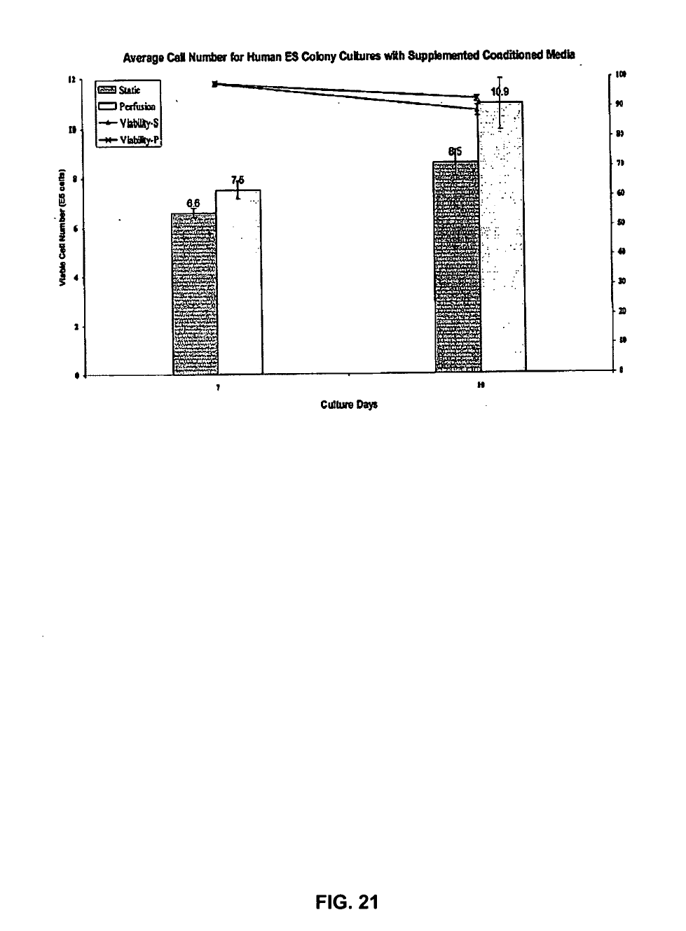

The graph in FIG. 21 shows the average cell numbers of two independent perfusion runs on human ES colony cultures. On Day 7, a 14% increase in cell density for perfusion (7.5×10⁵ cells/ml) compared to static (6.6×10⁵ cells/ml) was observed. The cell density of perfusion cultures increased further to 28% (10.9×10⁵ cells/ml) more than static (8.5×10⁵ cells/ml) by Day 10. The increase in cell density for day 10 for perfusion was significant at a 1-tail student t-test of 10% (p=0.079).

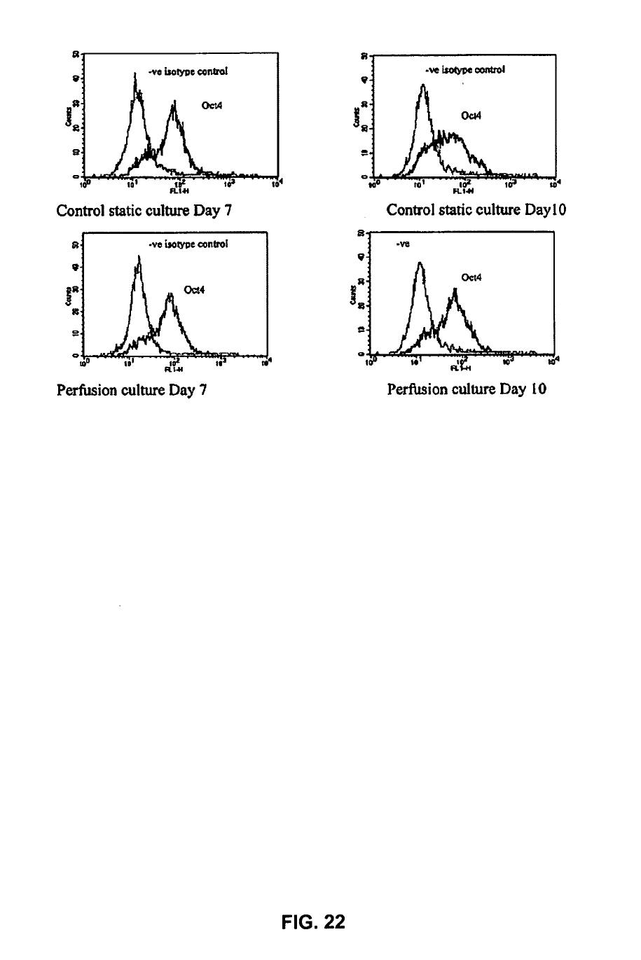

FIG. 22 shows the Oct 4 FACS results for perfused and static human ES colony cultures on Day 7 and 10, indicating a healthy expression of Oct4 in both cultures on Day 7 and 10, thus the cells remain pluripotent.

Perfusion Cultures of hES Cell Clumps on Organ Culture Dish

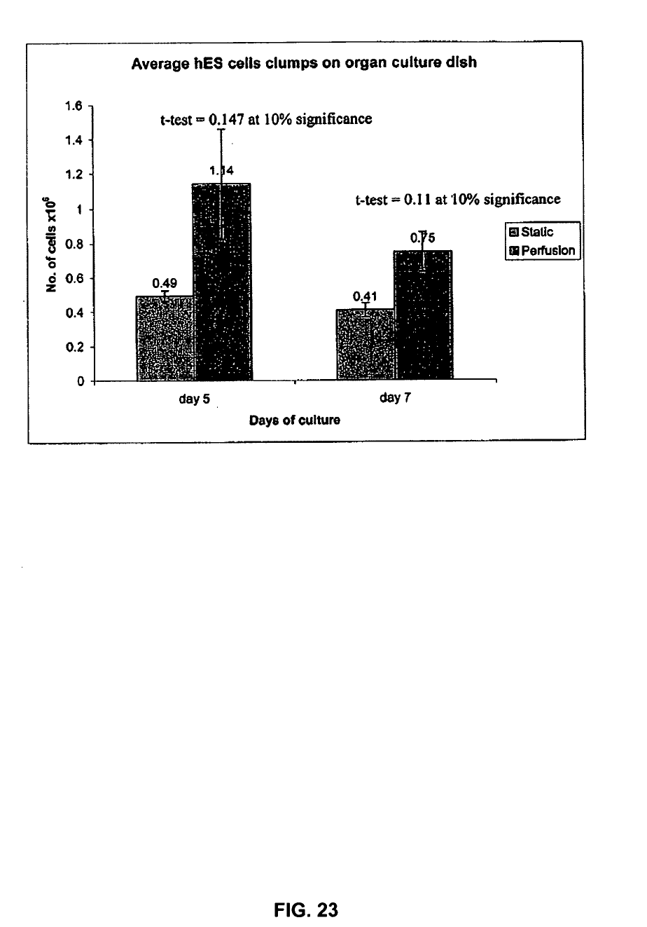

The graph of the average cell number of 2 separate perfusion runs is as shown in FIG. 23. As can be seen, there is a significant, more than 2-fold increase in total viable cells after 5 days of culture in perfusion. The normal static culture hardly grows any further and there is a slight decline in cell numbers on day 7. This decline is also seen for the perfusion culture, but cells in perfusion culture still perform better overall in the organ culture dish.

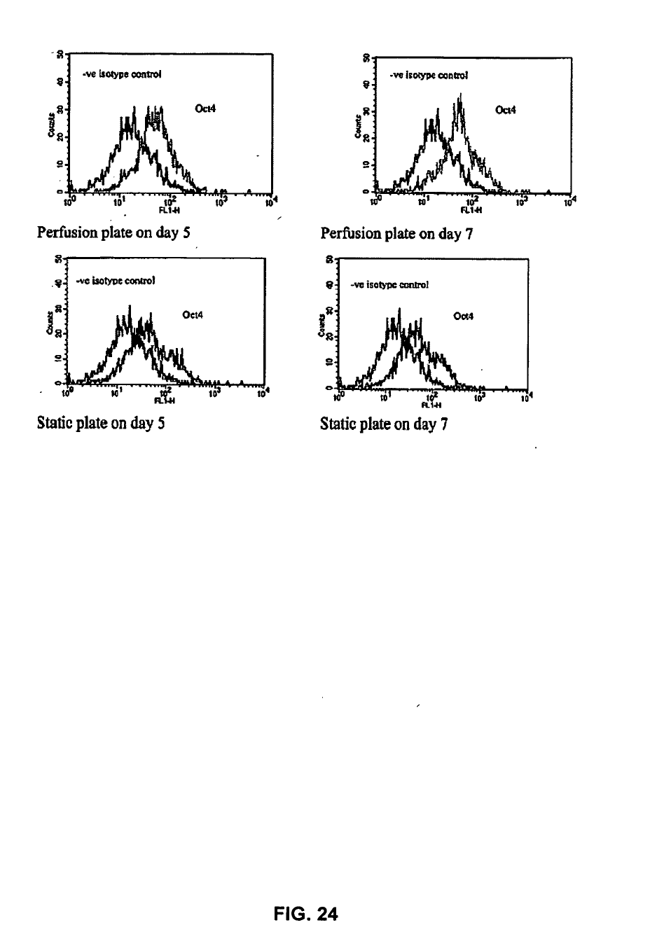

FIG. 24 shows the FACS results of the hES cells, indicating that Oct4 protein expression continues to be present in the cultures on days 5 and 7 and thus the cells continue to remain pluripotent.

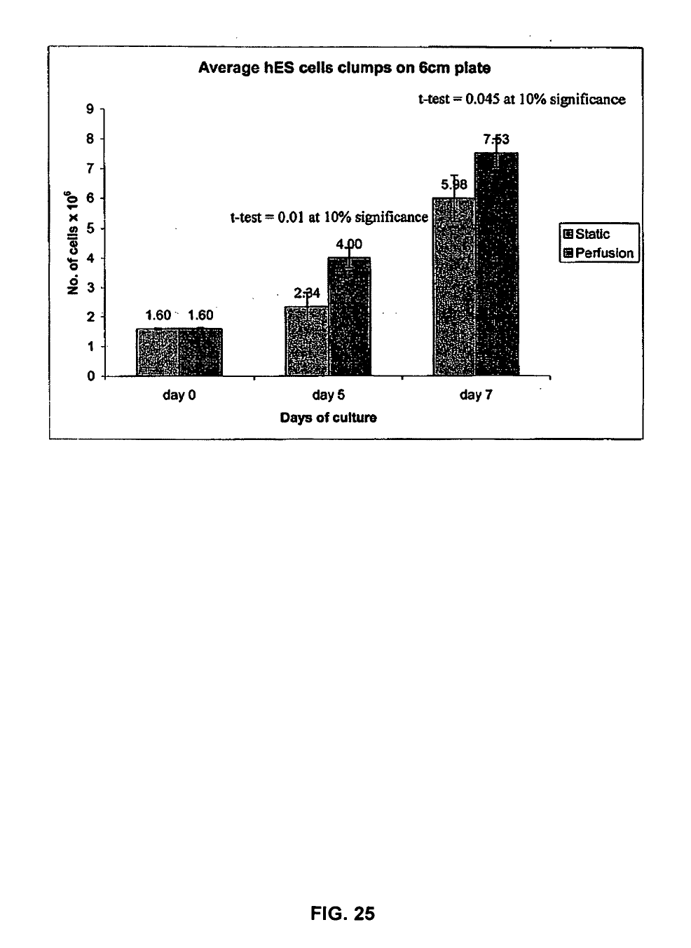

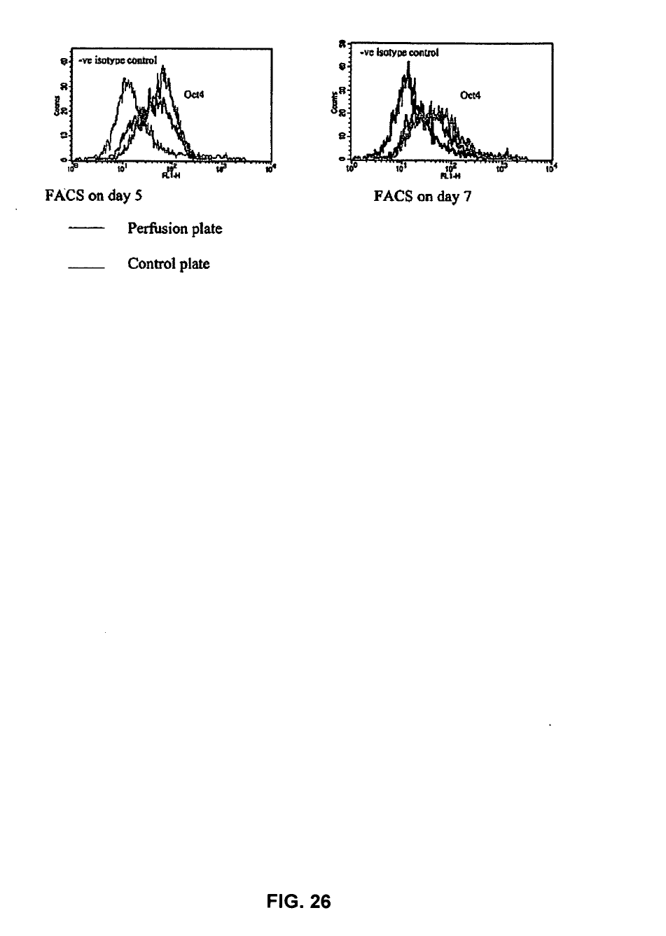

Perfusion cultures of hES cell clumps on 6 cm plates The graph of the average cell number of 2 separate perfusion runs at a larger scale in 6 cm petridishes is shown in FIG. 25. Once again, it can be seen that perfusion leads to almost 2-fold higher cell numbers than the static control cultures on day 5. By day 7, the relative increase in cell numbers is about 25% more than the control, perhaps due to a limitation in available surface area for further expansion. The FACS results of Oct4 protein expression are stable on both the 5^{th} and 7^{th} days when cells are harvested, showing that the expanded hES cells remain pluripotent.

From the results that were obtained with 3 different conditions, namely:—a) colony cultures in organ culture dishes b) clump cultures in organ culture dishes c) clump cultures in 6 cm Petri dishes, perfusion appears to provide a significant growth advantage when compared to static cultures.

Day 5 of the clump cultures show a much higher, about 2-fold greater, cell density in perfusion mode. Results obtained on day 7 show that cells continue to perform better in perfusion mode, however, the relative increase is less. This could be due to several issues:—
(1) The factors in the conditioned media may be insufficient near the end of the cultures to support exponential growth. In which case, the concentration of the conditioned media may have to be strengthened at the end of the culture;
(2) Insufficient surface area for cells to expand, limiting the effects of perfusion. Visually from the pictures taken of the culture dishes, cells looked rather confluent, thus surface limitation may be a real issue.
(3) Though not investigated here, seeding density of the culture may also be important. When a culture is seeded lower the effect of perfusion may be more pronounced as compared to when seeding is high so that a confluent plate would be obtained at the end of the 7 days. If the seeding density is high and confluent plates are achieved sooner, the limiting factor might be due to surface inhibition near the end of the culture, thus diminishing the effect of perfusion.

To overcome these limitations, hES cells should not be overseeded, and perfusion may be operated only for 5 days and they could be harvested earlier and seeded to fresh surfaces. In this way, more cells are harvested compared to the controls and the culture time shortened.

FACS results showed that the cells are still pluripotent when the cells are cultured in perfusion. Thus Oct-4 transcription factor expression is maintained in all 4 runs of perfusion, 2 on organ culture dishes and 2 on 6 cm plates.

Conclusions

The success of a perfusion bioreactor for mouse ES cell proliferation has been demonstrated and has important applications for human ES cell proliferation to produce therapeutic quantities of ES cells and to make sufficient cells available for differentiation into a variety of useful tissues.

Conclusions for Perfusion Culture of mouse embryonic stem (mES) cells on petriperm™ membrane

High density ES cell culture was obtained on the surface of a hydrophilic plastic membrane surface called petriperm™ or biofolie™.

Perfusion culture combined with growing embryonic cells on petriperm™ membrane enhances proliferation of ES cells by on average 9-fold more than if they were grown on petridishes in static culture. This significant increase in total cell number provides a major advantage in the provision of stem cells for therapeutic, diagnostic and research purposes.

Cell densities for all experiments involving petriperm™ membrane are much higher than that for conventional Petri dish culture. Performance of cell cultures followed the trend:—perfusion culture in petriperm™ dishes (9-fold increase)>static culture in petriperm™ dishes=static culture in petribags (each showing a 6-fold increase)>perfusion culture in petribags (5-fold)>>static culture in petri dishes.

Through the use of perfusion culture with petriperm™ dish to increase the proliferation of cultures of mouse embryonic stem cells, the inventor has shown that the cell viability is comparable to that of conventional petri dish culture while the viable cell density is improved by more than 9-fold. The various assays such as SSEA-1 and Oct-4, embryoid bodies development and teratoma formation in SCID mice, demonstrated that the mES cells cultured by coupling perfusion culture with the use of petriperm™ dish remain pluripotent and undifferentiated. This is the first example of a bioreactor which can significantly increase the densities of embryonic stem cell culture.

Improvements on the scale-up version may have to be made as petriperm™ bags appear to be less suitable for mES cells growth, though they still provide at least a 6-fold increase in cell density over conventional petri dish cultures. The reason is bags are more flimsy than the dishes, and consequently more easily creased than the dishes. However, petribags can be easily manufactured to increase the available volume and surface area for ES cell proliferation. To address this issue, the inventor recognizes that a taut petriperm™ membrane is required and thus, he has designed prototypes of feasible scale-up systems. Examples of 2 prototypes have been designed as shown in FIGS. 20a and 20b; both versions will allow stacking of the cultures one on top of another as a means of increasing the surface area available for cell growth. Both 50 cm² and 100 cm² surface area designs are available.

Conclusions For Perfusion Culture Of Human Embryonic Stem Cells Compared To Control Cultures Without Perfusion

The inventors have also demonstrated the effective use of perfusion culture systems in increasing the proliferation of human embryonic stem cell cultures. 2× conditioned media was prepared by 24 hour incubation of growth inactivated support cells, such as mitotically inactivated mouse embryonic fibroblasts, with culture media. The incubated media was then diluted, volume for volume, with non-incubated culture media. This 1× supplemented conditioned medium was used by the inventors to obtain the improved human ES cell yield. Although not wishing to be bound by any theory, the inventors consider that this effect may be due to an increase in growth factor concentration in the incubated medium, wherein the subsequent dilution ensures that sufficient space and resources are provided for the extra cell growth. Interestingly, when cells were incubated to much higher concentrations, the subsequently diluted conditioned medium did not provide a significant increase in human ES cell growth suggesting that a reasonably fine balance exists in which increased human ES cell growth may occur.

### REFERENCES

1. Chu L, Robinson DK. 2001. Industrial choices for protein production by large-scale cell culture. Curr Opin Biotechnol 12:180-187
2. Ling V, Neben S. 1997. In vitro differentiation of embryonic stem cells: immunophenotypic analysis of cultured embryoid bodies. J Cell Physiol 171:104-115
3. Murray, Patricia; Edgar, David. 2001. The regulation of embryonic stem cell differentiation by leukaemia inhibitory factor. Differentiation Vol 68, 4-5:227-234
4. Niwa H., J.-I.Miyazaki, and A. G. Smith, 2000. Quantitative expression of Oct3/4 defines differentiation, dedifferentiation or self-renewal of ES cells. Nat. Genet. 24:372-376
5. Oka, Masahiro; Tagoku, Kenichi; Russell, Thomas L.; Nakano, Yuka; Hamazaki, Takashi; Meyer, Edwin M.; Yokota, Takashi; Terada, Naohiro.2002. CD9 is associated with leukemia inhibitory factor-mediated maintenance of embryonic stem cells. Mol Biol Cell Vol 13, 4:1274-1281
6. Robertson M, Chambers I, Rathjen P, Nichols J, Smith A. 1993. Expression of alternative forms of differentiation inhibiting activity (DIA/LIF) during murine embryogenesis and in neonatal and adult tissues. Dev Genet 14:165-173
7. Sowmya Viswanathan, Tania Benatar, Stefan Rose-John, Doug A. Lauffenburger, Zandstra P. W. 2002. Ligand/Receptor signaling threshold (LIST) model accounts for gp130-mediated embryonic stem cell self-renewal responses to LIF and HIL-6. Stem Cell 20:119-138
8. Tae Gwan Park. Perfusion culture of hepatocytes within galactose-derived biodegradable poly (lactide-co-glycolide) scaffolds prepared by gas foaming of effervescent salts. January 2002. Journal of Biomedical Materials Research Vol 59, Issue 1, Pages 127-135
9. Williams M A, Gross S K, Evans J E, McCluer RH. 1988. Glycolipid stage-specific embryonic antigens (SSEA-1) in kidneys of male and female C57BL/6J and beige adult mice. J Lipid Res 29:1613-1619
10. Zandstra P W, H.-V. Le, G. Q. Daley, L. G. Griffith, D. A. Lauffenburger, 2000. Leukemia Inhibitory Factor (LIF) concentration modulates embryonic stem cell self-renewal and differentiation independently of proliferation. Biotechnol and Bioeng, vol. 69 No. 6: 607-617
11. Thomson J. A., Itskovitz-Eldor J., Shapiro S. S., Waknitz M. A., Swiergiel J. J., Marshall V. S., Jones J. M., 1998. Embryonic Stem Cell Lines Derived from Human Blastocysts. Science Vol 282, 1145-1147.

1. A method of generating stem cells from a stem cell culture comprising the steps of:
(i) attaching one or a plurality of stem cells to the surface of a membrane; and
(ii) perfusing the stem cell(s) with culture media.

2. The method of claim 1 wherein said stem cell culture is perfused with culture media for one or more predetermined time periods.

3. The method of claim 2 wherein the stem cell culture is perfused with culture media for a single time period.

4. The method of claim 2 wherein the stem cell culture is perfused with culture media for a plurality of time periods.

5. The method of claim 2 wherein the stem cell culture is perfused with culture media for a plurality of time periods, wherein each time period is of the same length.

6. The method of claim 2 wherein the stem cell culture is perfused with culture media for a plurality of time periods, wherein the time periods are separated by intervals of between 1 and 24 hours.

7. The method of claim 2 wherein the stem cell culture is perfused for one or a plurality of first time periods and for one or a plurality of second time periods, wherein said first and second time periods are of different lengths.

8. The method of claim 1 wherein the flow of culture media is a pulsed flow.

9. The method of claim 1 wherein the stem cell culture is perfused with between 0.05 and 5 ml culture media per minute.

10. The method of claim 1 further comprising the step of culturing the stem cells in non-perfusion culture for a first culture period prior to the step of perfusing the stem cell culture.

11. The method of claim 1 further comprising the step of attaching stem cells to a membrane surface prior to the step of perfusing the stem cell culture.

12. The method of claim 11 wherein said membrane is hydrophilic.

13. The method of claim 11 wherein said membrane is gas permeable.

14. The method of claim 11 wherein said membrane is made from a plastics material.

15. The method of claim 11 wherein said membrane is a petriperm™ membrane.

16. The method of claim 1 wherein said membrane forms a bag or pouch, the stem cells attached to the interior of the bag or pouch.

17. The method of claim 1 further comprising the step of collecting generated stem cells.

18. The method of claim 1 wherein said stem cells are selected from the group consisting of:
embryonic stem cells;
mammalian embryonic stem cells;
mouse embryonic stem cells;
human embryonic stem cells.

19. A pharmaceutical composition comprising stem cells generated by the method of claim 1, or fragments or products thereof.

20. A method of medical treatment comprising administering to an individual a therapeutically effective amount of a pharmaceutical composition of claim 19.

21. Apparatus for generating stem cells from a stem cell culture by perfusing the stem cells with culture media, the apparatus comprising:
a membrane configured for attachment of one or more stem cells to be cultured;
culture media supply means for supplying culture media to the stem cell culture;
culture media removal means for removing culture media from said stem cell culture,
wherein said supply and removal means are arranged to flow culture media through the stem cell culture.

22. Apparatus as claimed in claim 21 wherein the supply and removal means are pumps, the removal means arranged to operate at a higher frequency than the supply means.

23. Apparatus as claimed in claim 21 wherein the supply and removal means generate a perfusion flow rate of culture media in the range 0.05 to 5 ml/min.

24. Apparatus for culturing stem cells comprising:
(a) a housing having a wall defining an internal chamber;
(b) a stem cell culture membrane extending across said chamber and held in position by;
(b) membrane support means; and further comprising
(c) means to supply culture media to said membrane.

25. Apparatus as claimed in claim 24 wherein said membrane is hydrophilic.

26. Apparatus as claimed in claim 24 said membrane is gas permeable.

27. Apparatus as claimed in claim 24 wherein said membrane is made from a plastics material.

28. Apparatus as claimed in claim 24 wherein said membrane is a petriperm™ membrane.

29. Apparatus as claimed in claim 24 wherein said membrane is gas permeable and comprises a first hydrophilic cell culture surface and a second surface, said apparatus further comprising means to deliver a gas supply to said second surface, wherein said means to supply culture media is configured to supply culture media to said first hydrophilic cell culture surface.

30. Apparatus as claimed in claim 24 wherein said housing comprises a first engagement portion at a first end of said housing and a second engagement portion at a second end of said housing, said first or second engagement portion configured to engage with the other said portion of another said apparatus.

31. A method of preparing a conditioned medium comprising the steps of:
(a) culturing support cells in a culture media;
(b) removing a quantity of the culture media; and
(c) diluting the removed culture media.

32. The method of claim 31 wherein said dilution is with culture media of the kind used in step (a).

33. The method of claim 31 wherein said support cells are embryonic fibroblasts.

34. The method of claim 31 wherein said support cells are mouse embryonic fibroblasts.

35. The method of claim 31 wherein the support cells are cultured in step (a) for at least 12 hours.

36. The method of claim 31 wherein the support cells are cultured in step (a) for up to 48 hours.

37. The method of claim 31 wherein the support cells in step (a) are cultured for between about 22-26 hours.

38. The method of claim 31 wherein the support cells in step (a) are cultured for about 24 hours.

39. The method of claim 31 wherein said support cells are mitotically inactivated.

40. The method of claim 31 further comprising the step of treating the support cells with mitomycin.

41. The method of claim 31 wherein in step (a) the support cells are cultured at a density of 1-5×10⁵ cells/ml.

42. The method of claim 31 wherein in step (a) the support cells are cultured at a density of 2-4×10⁵ cells/ml.

43. The method of claim 31 wherein in step (a) the support cells are cultured at a density of about 3.5×10⁵ cells/ml.

44. The method of claim 31 further comprising the step of:
(d) culturing stem cells in the conditioned medium.

45. The method of claim 44 wherein said stem cells are selected from the group consisting of:
embryonic stem cells;
mammalian embryonic stem cells;
mouse embryonic stem cells;
human embryonic stem cells.

46. The method of claim 44 wherein said culturing step (d) comprises perfusion culture.

47. The method of claim 44 wherein said culturing step (d) comprises static culture.

48. The method of claim 31 further comprising the step of:
(d) culturing stem cells in the conditioned medium, wherein said stem cells are attached to a membrane.

49. The method of claim 48 wherein said membrane is hydrophilic.

50. The method of claim 48 wherein said membrane is gas permeable.

51. The method of claim 48 wherein said membrane is made from a plastics material.

52. The method of claim 48 wherein said membrane is a petriperm™ membrane.
